(19) United States
(12) Patent Application Publication
(10) Pub. No.: US 2019/0216628 A1
(43) Pub. Date: Jul. 18, 2019

(54) APPARATUS FOR DAMPING INVOLUNTARY HAND MOTIONS

(71) Applicant: Steadiwear Inc., Toronto (CA)

(72) Inventors: Mark Elias, Toronto (CA); Swapnil Patel, Toronto (CA); Emile Maamary, Toronto (CA); Leonardo Araneta, Toronto (CA); Numaira Obaid, Mississauga (CA)

(73) Assignee: Steadiwear Inc.

(21) Appl. No.: 16/360,366

(22) Filed: Mar. 21, 2019

Related U.S. Application Data
(63) Continuation of application No. PCT/CA2017/051087, filed on Sep. 15, 2017.
Application No. PCT/CA2017/051087 is a continuation-in-part of application No. 15/459,513, filed on Mar. 15, 2017.
(60) Provisional application No. 62/397,994, filed on Sep. 22, 2016.
(60) Provisional application No. 62/397,994, filed on Sep. 22, 2016.
(60) Provisional application No. 62/308,419, filed on Mar. 15, 2016.

(51) Int. Cl. A61F 5/01; F16F 9/00; F16F 9/30; F16F 9/32; F16F 9/504; F16F 9/53

(52) U.S. Cl. CPC A61F 5/013; F16F 9/006; F16F 9/303; F16F 9/3214; A61F 2005/0155; F16F 9/53; A61F 2005/0179; A61F 2005/0165; F16F 9/504

(57) ABSTRACT

An apparatus for damping involuntary hand motions occurring in at least first and second directions is provided, the first direction being opposite the second direction. The apparatus is securable to a hand and a forearm and comprises at least a first body having a terminal movable in an internal space in the body. The body contains a Non-Newtonian fluid. A link connects the terminal to a first portion of the apparatus.

### FIELD

The present invention relates to devices for damping involuntary hand motions.

### INTRODUCTION

The following paragraphs are not an admission that anything discussed in them is prior art or part of the knowledge of persons skilled in the art.

A percentage of the world's population suffers from involuntary hand or forearm motions such as hand or elbow tremors. Involuntary hand and forearms motions may involve rhythmic muscle movement resulting in hand or forearm oscillation. There are many types of involuntary hand and forearm motions. Involuntary hand and forearm motions may vary in amplitude, frequency, and may occur in one or more directions. A majority of involuntary hand and forearm motions have been found to occur at a frequency ranging between 2 hz and 12 hz. Involuntary hand and forearm motions may be associated with Parkinson's disease and Essential Tremor. Depending on severity, involuntary hand motions may impede daily activities and reduce quality of life of the persons experiencing the involuntary hand motions. Devices may be worn by these persons to damp the involuntary hand motions. Such devices may be generally classified as passive and active systems.

Passive systems may use mechanical linkages frictionally bound together to make it more difficult for a user to move their hand and thereby reduce the amplitude with which the hand may otherwise move or oscillate. Passive systems may be bulky and uncomfortable to wear, and may provide suboptimal involuntary hand motion damping performance depending on the particular type of involuntary hand motions experienced by the user. One limitation of passive systems may be that passive systems may not be capable of adapting to varying loading conditions.

Active systems may overcome this limitation by using active force feedback mechanisms which may sense the dynamics of involuntary hand motions and generate involuntary hand motion damping forces in response to the sensed dynamics. Such mechanisms require a power source and may include batteries, sensors, rotary drives, magnetic field generators, and gyroscopes. The use of electronics imports limitations. For example, power sources such as batteries may be relatively heavy and may need to be recharged frequently and replaced periodically. Such limitations may make devices implementing active force feedback systems less convenient to use and more expensive to buy and maintain. Also, active systems may be sensitive to water, magnetic fields, temperature changes, and shocks that may result when a user accidentally drops or hits the device against a hard surface.

There are seven degrees of freedom in arm motion: 1. shoulder rotation, 2. shoulder flexion/extension, 3. shoulder abduction/adduction, 4. elbow pronation/supination, 5. elbow flexion/extension, 6. wrist flexion/extension, and 7. wrist radial/ulnar deviation. Research has found that the majority of tremors fall into the following three degrees of freedom: wrist flexion/extension, wrist radial/ulnar deviation and elbow pronation/supination. It is of critical importance for a device to dampen motion in these three degrees of freedom for the function of the unit as a whole to be comprehensive.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

Throughout this disclosure a body is described as affixed to a second portion of an apparatus and connected to a first portion via a link or connector. This reference is for simplicity, and should not be understood as limiting the application, which also contemplates a configuration in which a body is affixed to a first portion of an apparatus and is connected to a second portion via a link or connector.

In a first embodiment, there is provided an apparatus for damping involuntary hand motions occurring in at least first and second directions, the first direction being opposite the second direction. The apparatus comprises a first portion securable to a hand, a second portion securable to a forearm and movable relative to the first portion, and a body secured to the second portion. The body comprises an exterior surface, an interior space containing a Non-Newtonian fluid, and a port extending from the exterior surface into the interior space. The body further comprises a terminal in the interior space movable between the first and the second positions and biased toward the first position, the first position being located farther away from the first portion than the second position, and a link passing through the port and connecting the terminal to the first portion. When the first portion is secured to the hand and the second portion is secured to the forearm, the link is in tension, and the involuntary hand motions in the first direction move the terminal from the first position toward the second position.

In some versions of this first embodiment, the link of the apparatus is configured to oppose lengthwise tensile forces and to substantially not oppose lengthwise compression forces, such as the lengthwise tensile and compression forces as may be applied to the link by the involuntary hand motions.

In some versions of this first embodiment, the Non-Newtonian fluid has a composition selected to achieve a damping performance profile of the body optimized for damping involuntary hand motions ranging in frequency from 2 to 12 hertz.

In some versions of this first embodiment, the terminal of the body divides the interior space of the body into two sections and comprises a resistance flow opening connecting the first and second sections for bidirectional flow of the Non-Newtonian fluid, and a check valve connecting the first and second sections for unidirectional flow of the Non-Newtonian fluid.

In some versions of this first embodiment, the apparatus further comprises a tuned mass damper for damping involuntary forearm motions. The tuned mass damper comprises a housing securable to the second portion. The housing defines an interior space which includes a first interior wall positioned opposite to a second interior wall and a third interior wall positioned opposite to a fourth interior wall. The tuned mass damper further comprises an eccentric mass in the interior space. The eccentric mass extends between the first interior wall and the second interior wall, dividing the interior space adjacent to the eccentric mass into a first portion and a second portion. The tuned mass damper further comprises a fluid which is added to a remaining volume of the interior space. The eccentric mass comprises at least one canal to permit the fluid to flow between the first portion and the second portion. The eccentric mass is free to move out of phase with involuntary forearm oscillations in a linear direction between the third interior wall and the fourth interior wall.

In some versions of this first embodiment, the eccentric mass is biased in a central region of the interior space by a plurality of springs that connect the third interior wall with the eccentric mass and by a plurality of springs that connect the fourth interior wall with the eccentric mass such that the eccentric mass moves out of phase with involuntary forearm oscillations in a linear direction between the third interior wall and the fourth interior wall.

In a second embodiment, there is provided an apparatus similar to the apparatus described as a first embodiment above except insofar as the body of this apparatus is a first body and this apparatus has a second body that is the same as the first body and is secured to the second portion similar to the first body. The link of the second body is connected to the first portion similar to the link of the first body. When the first portion is secured to the hand and the second portion is secured to the forearm, the link of the second body is in tension, and the involuntary hand motions in the second direction move the terminal of the second body from the first position of the terminal of the second body toward the second position of terminal of the second body.

In a third embodiment, there is provided an apparatus for damping involuntary hand motions. The apparatus comprises a first portion securable to a hand, a second portion securable to a forearm and movable relative to the first portion, and at least one body. The body comprises a casing having an exterior surface and defining a spherical interior space, and having a port extending from the exterior surface into the spherical interior space, the casing connected to the second portion. The body also comprises a connector, the connector comprising a spherical terminal filling the spherical interior space save for a gap between the spherical terminal and a wall of the spherical interior space, and a link passing through the port and connecting the spherical terminal to the first portion. The first portion may be secured to the hand and the second portion secured to the forearm, and the involuntary hand motions in either first or second directions may rotate the spherical terminal within the interior space and cause sheer friction between the spherical terminal and the shear thickening fluid.

In some versions of this third embodiment, the link comprises a telescoping cylinder with one main stage and at least one additional stage.

In some versions of this third embodiment, the link comprises a shaft and a linear bearing.

In some versions of this third embodiment, the link comprises a linear ball bearing.

In some versions of this third embodiment, the gap between the spherical terminal and the wall of the spherical interior space is between 0.0746 mm and 1.0254 mm wide.

In some versions of this third embodiment, the shear thickening fluid has a composition selected to resist involuntary hand motions above 3 hertz.

In some versions of this third embodiment, the shear thickening fluid has a composition selected to resist involuntary hand motions above 2 hertz.

In some versions of this third embodiment, the shear thickening fluid has a composition selected not to resist hand motions below 1 hertz.

In some versions of this third embodiment, the shear thickening fluid has a composition selected not to resist hand motions below 2 hertz.

In some versions of this third embodiment, the body further comprises an elastic o-ring positioned between the casing and the spherical terminal to hinder the passage of shear thickening fluid through the port.

In some versions of this third embodiment, the spherical terminal is configured to include an outer surface comprising a plurality of cavities.

In some versions of this third embodiment, the apparatus further comprises a tuned mass damper for damping involuntary forearm motions. The tuned mass damper comprises a housing securable to the second portion. The housing defines an interior space which includes a first interior wall positioned opposite to a second interior wall and a third interior wall positioned opposite to a fourth interior wall. The tuned mass damper further comprises an eccentric mass in the interior space. The eccentric mass extends between the first interior wall and the second interior wall, dividing the interior space adjacent to the eccentric mass into a first portion and a second portion. The tuned mass damper further comprises a fluid which is added to a remaining volume of the interior space. The eccentric mass comprises at least one canal to permit the fluid to flow between the first portion and the second portion. The eccentric mass is free to move out of phase with involuntary forearm oscillations in a linear direction between the third interior wall and the fourth interior wall.

In some versions of this third embodiment, the eccentric mass is biased in a central region of the interior space by a plurality of springs that connect the third interior wall with the eccentric mass and by a plurality of springs that connect the fourth interior wall with the eccentric mass such that the eccentric mass moves out of phase with involuntary forearm oscillations in a linear direction between the third interior wall and the fourth interior wall.

Further aspects and advantages of the embodiments described herein will appear from the following description taken together with the accompanying drawings.

### BRIEF DESCRIPTION OF THE FIGURES

For a better understanding of the described embodiments and to show more clearly how they may be carried into effect, reference will now be made, by way of example, to the accompanying drawings in which:

FIG. 1 is an isometric view of an example apparatus for damping involuntary hand motions, according to a variation of a first embodiment, secured to a hand and a forearm, with the hand being held in a neutral position;

FIG. 2 is a view of a ventral (palm) side of the hand and the forearm, and of an example of an apparatus for damping involuntary hand motions, according to a variation of a second embodiment, secured to the hand and the forearm;

FIG. 3 is an isometric assembly view of an example body of the example apparatuses of FIGS. 1 and 2;

FIG. 4 is a top section view of the example body of FIG. 3, showing a terminal of the example body, the terminal being in a first position;

FIG. 5 is a top section view of the example body of FIGS. 3 and 4, showing the terminal of the example body being in a second position;

FIG. 6 is an isometric view of the example apparatus of FIG. 1, shown with the hand held in a fully-flexed position;

FIG. 7 is an isometric view of the example apparatus of FIG. 1, shown with the hand held in a fully-extended position;

FIG. 8A corresponds to the example apparatus shown in FIG. 6, and shows some internal features of said apparatus, for clarity;

FIG. 8B corresponds to the example apparatus shown in FIG. 6, and shows some internal features of said apparatus, for clarity;

FIG. 9 is a side view of an apparatus for damping involuntary hand motions, according to a variation of a second embodiment, secured to the hand and the forearm;

FIG. 10 is an isometric view of a connector for connecting a link of any one of the example apparatuses of FIGS. 1 to 9 to a first portion of the apparatus of any one of FIGS. 1 to 9;

FIG. 11 is a damping performance profile of a terminal of the apparatus of FIGS. 1 to 10; and

FIG. 12 is a viscosity performance profile of a Non-Newtonian fluid that may be used to achieve the damping performance profile of FIG. 11.

FIG. 13 is an isometric view of an example apparatus for damping involuntary hand motions, according to a first variation of a third embodiment, secured to a hand and a forearm, with the hand being held in a neutral position;

FIG. 14 is an isometric assembly view of an example body of the example apparatus of FIG. 13;

FIG. 15 is a cross sectional isometric view of the example apparatus of FIG. 13;

FIG. 16 is an isometric view of the example apparatus of FIG. 13, shown with the hand held in a fully-flexed position;

FIG. 17 is an isometric view of the example apparatus of FIG. 13, shown with the hand held in an extended position;

FIG. 18 is a cross sectional view of the body portion of the example apparatus of FIG. 13;

FIG. 19 is an example damping performance profile of a terminal of the apparatus of FIGS. 13 to 18;

FIG. 20 is an isometric view of an example apparatus for damping involuntary hand motions, according to a second variation of a third embodiment, secured to a hand and a forearm, with the hand being held in a neutral position;

FIG. 21 is an isometric assembly view of an example body of the example apparatus of FIG. 20;

FIG. 22 is a cross sectional isometric view of the example apparatus of FIG. 20;

FIG. 23 is an isometric view of the example apparatus of FIG. 20, shown with the hand held in a flexed position;

FIG. 24 is an isometric view of the example apparatus of FIG. 20, shown with the hand held in an extended position;

FIG. 25 is an example damping performance profile of a terminal of the apparatus of FIGS. 20 to 24.

FIG. 26A is an isometric view of an example spherical terminal attached to a link.

FIG. 26B is a side view of the example spherical terminal attached to the link shown in FIG. 26A.

FIG. 27A is a top view of the example spherical terminal shown in FIGS. 26A to 26B;

FIG. 27B is a rear view of the example spherical terminal shown in FIG. 27A;

FIG. 28 is an isometric view of an example tuned mass damper for dampening involuntary forearm motions connected to an example apparatus for dampening involuntary hand motions, in accordance with an embodiment;

FIG. 29 is an isometric assembly view of the example tuned mass damper of FIG. 28;

FIG. 30 is an isometric view of the apparatus of FIG. 28 with a top cover removed from the example tuned mass damper; and

FIG. 31 is an isometric view of the apparatus of FIG. 30 with an eccentric mass in a stabilizing position.

### DETAILED DESCRIPTION

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments generally described herein. Furthermore, this description is not to be considered as limiting the scope of the embodiments described herein in any way, but rather as merely describing the implementation of various embodiments as described.

Throughout this disclosure the body is depicted and described as affixed to the second portion of the apparatus, and connected to the first portion via a link. This reference is for simplicity, and should not be understood as limiting the application, which also contemplates a configuration in which the body is affixed to the first portion of the apparatus and is connected to the second portion via a link.

Various embodiments of an apparatus for damping involuntary hand motions are described below. As will be described below, the apparatus may use Non-Newtonian fluid in combination with the various features of the apparatus to provide damping forces for damping the involuntary hand motions, which damping forces may vary in response to variations of one or more of frequency and amplitude of said motions, according to at least one pre-determined damping performance profile. A Non-Newtonian fluid, is a fluid at least some of the properties of which may differ from the properties of Newtonian fluids. For example, the viscosity of Non-Newtonian fluid may vary with varying loading applied to the Non-Newtonian fluid. In particular, the viscosity of the Non-Newtonian fluid may vary with at least the frequency of the loading applied to the fluid. A non-Newtonian fluid may include a shear thickening fluid.

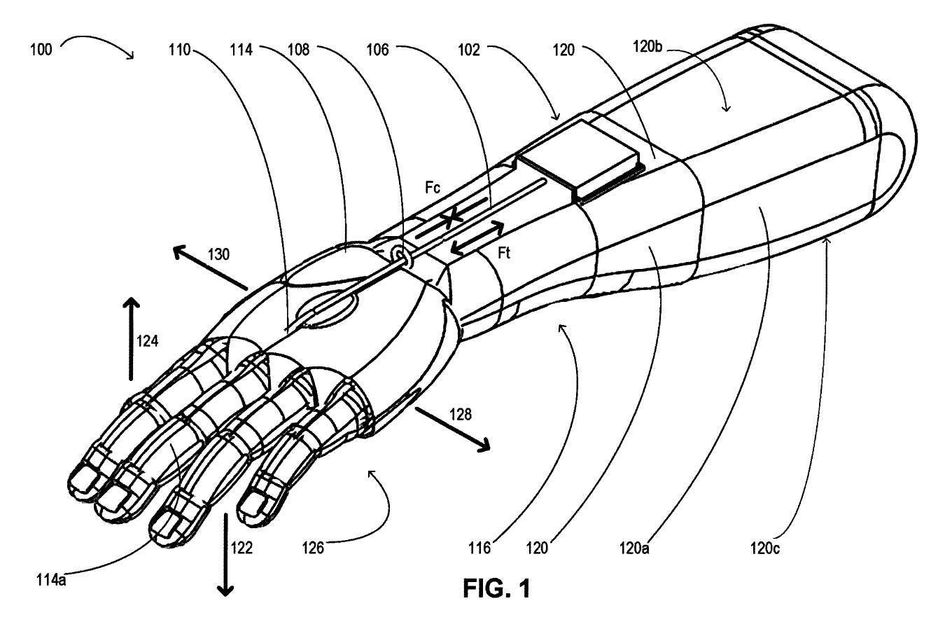

Reference is now made to FIG. 1, depicting an apparatus according to a variation of a first embodiment. The involuntary hand motions may occur in at least a first and second direction 122, 124, the first direction 122 being opposite the second direction 124. Involuntary hand motions may also occur in other directions 130 and 128. FIG. 1 shows an example of an apparatus 100 for damping involuntary hand motions occurring in at least a first 122 and second 124 directions, the first direction 122 being opposite the second direction 124. As shown, the apparatus 100 may comprise a first portion 114 securable to a hand 114a and a second portion 120 securable to a forearm 120a and movable relative to the first portion 114. The apparatus 100 may further comprise a body 102 secured to the second portion 120. Said body 102 may damp the involuntary hand motions in one of the at least first 122 and second 124 directions.

In some embodiments, the body 102 may be permanently secured to the second portion 120. In other embodiments, the body 102 may be removably secured to the second portion 120. A permanent securement may be a securement designed to not be altered by a user of the apparatus 100 after manufacturing and assembly of the apparatus 100. A removable securement may include mating portions, at least one on each of the second portion 120 and the body 102. The mating portions may hold the body 102 in place for operation of the apparatus 100 and may allow for the user or a technician to detach the body 102 from the second portion 120 for maintenance or replacement. In some embodiments, the removable securement may be configured to allow for adjusting the position of the body 102 relative to the second portion 120. Any suitable combination of known methods and components may be used to implement any one or a combination of the permanent and removable securements.

FIG. 1 shows the hand 114a being held in a neutral position, and shows the hand 114a and the forearm 120a each having a dorsal side 120b and a ventral side 120c. As shown, the apparatus 100 may include only one body 102. The body 102 may be positioned relative to the first and second portions 114, 120 such that when said portions 114, 120 are secured to the hand 114a and the forearm 120a, respectively, the body 102 is positioned over the dorsal side 120b of the hand 114a and the forearm 120a and may dampen involuntary hand motions that may occur in the first direction 122.

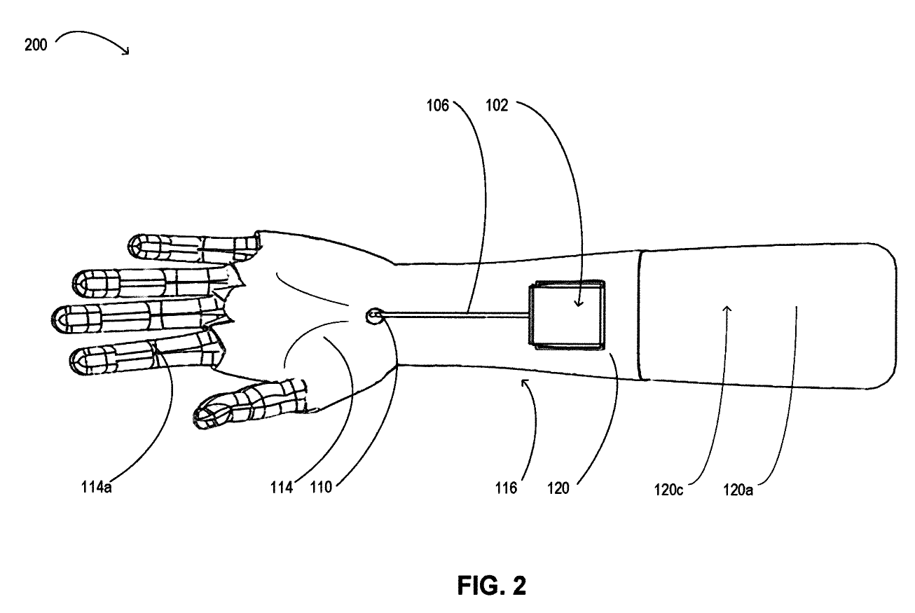

Likewise, as shown in FIG. 2, the body 102 may be positioned relative to the first and second portions 114, 120 such that when said portions 114, 120 are secured to the hand 114a and the forearm 120a, respectively, the body 102 is positioned over the ventral side 120c of the hand 114a and the forearm 120a and may dampen involuntary hand motions that may occur in the second direction 124.

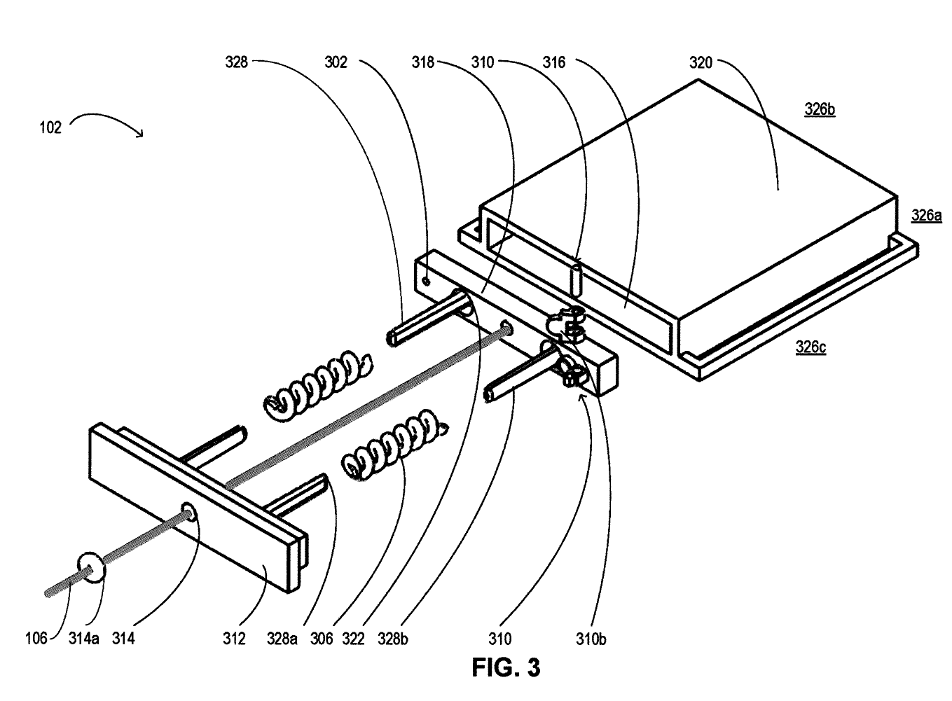

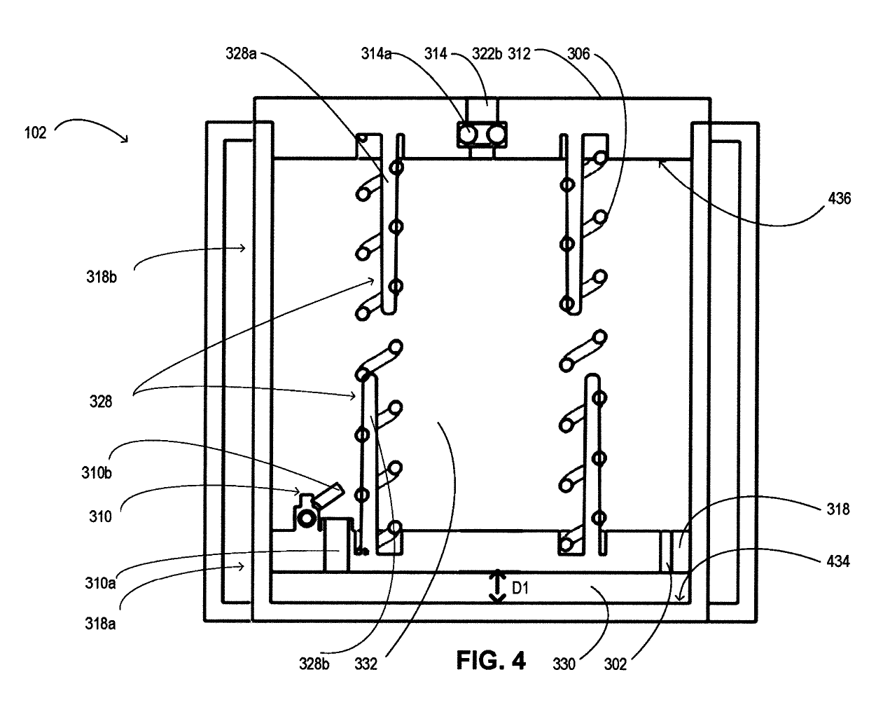

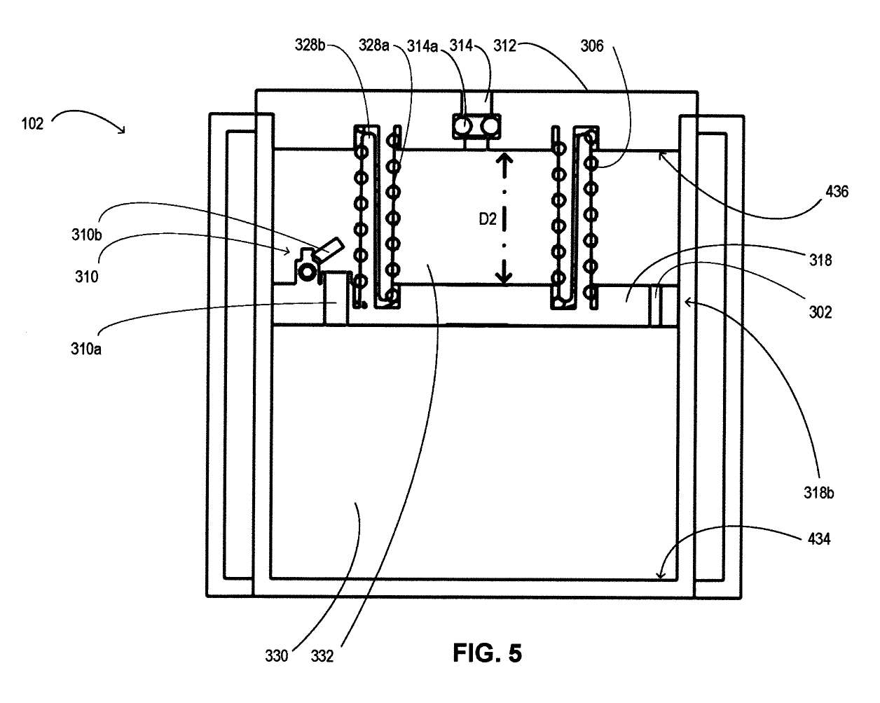

Reference is now made to FIGS. 3 to 5. The body 102 may comprise an exterior surface 320, an interior space 316 containing a Non-Newtonian fluid (not shown), and a port 314 extending from the exterior surface 320 into the interior space 316. The body 102 may further comprise a terminal 318 in the interior space 316 movable between a first 318a and second 318b positions and biased toward the first position 318a, the first position 318a being located farther away from the first portion 114 than the second position 318b. The body 102 may also comprise a link 106 passing through said port 314 and connecting the terminal 318 to the first portion 114.

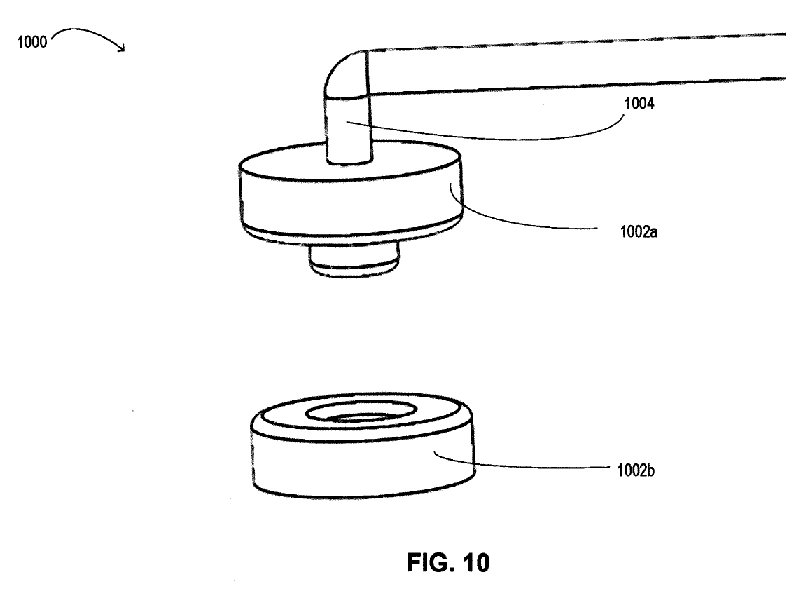

Reference is now briefly made to FIG. 10. The link 106 may connect the terminal 318 to the first portion 114 via any suitable connection. As shown in FIG. 10, the link 106 may have a distal portion 1004 that is farthest away from the body 102, 902 corresponding to that link 106 and a connector 1000 extending from that distal portion 1004. As shown, the connector 1000 may be a riveted button having a first 1002a and second 1002b mating portions that may engage each other removably or permanently. The first mating portion 1002a may extend from the distal portion 1004 of the link 106. The second mating portion 1002b may be secured to the first portion 114. As shown in FIG. 1, other suitable connections may include, for example, a permanent connection 110 between the link 106 and the first portion 114. The permanent connection 110 may be implemented by any suitable means. For example, by sewing or stitching the link 106 to the first portion 114.

As shown in FIG. 1, when the first portion 114 is secured to the hand 114a and the second portion 120 is secured to the forearm 120a, the link 106 may be in tension, and the involuntary hand motions in the first direction 122 may move the terminal 318 from the first position 318a toward the second position 318b. The link 106 may be configured to oppose lengthwise tensile forces Ft and to substantially not oppose lengthwise compression forces F_{c}, such as may be applied to the link 106 by the involuntary hand motions occurring in the first 122 and second 124 directions, respectively. The link 106 may cooperate with the terminal 318 and the Non-Newtonian fluid of the body 102 to dampen the involuntary hand motions in the first direction 122.

In some embodiments, the link 106 may be a tension-only link. That is, the link 106 may cooperate with the terminal 318 in lengthwise tension and substantially not in lengthwise compression. This functionality may be achieved in more than one way. For example, the link 106 may include a cable. The cable 106 may be selected to withstand lengthwise tensile forces applied to the cable 106 by the involuntary hand motions, and to bend and therefore exert substantially no reactive forces onto the terminal 318 when the involuntary motions apply lengthwise compression forces to the cable 106.

In other embodiments, the tension-only link 106 may include any other suitable combination of any one or more of flexible and rigid components, so long as that combination may result in similar tension-only operation. For example, one suitable combination (not shown) may include a pair of elongate members slidable one within the other along a length of each of the elongate members, with the range of the sliding motion being limited by one or more projections extending from one or both of the elongate members.

Many materials can withstand larger tensile forces than compression forces with a given geometry. Tension-only links 106 may be less susceptible to fatigue failure than tension-and-compression links. Therefore, tension-only links 106 may in some cases be made thinner than tension-and-compression links if tension-and-compression links were used in the apparatus 100, without reducing the lifespan of the links 106. In embodiments of the apparatus 100 where the link 106 is a flexible link 106, such as a cable made of a flexible plastic (in contrast to example links 106 comprising a pair of rigid sliding members providing the tension-only operation, as described in the foregoing paragraph), flexibility of the link 106 may allow for a simpler construction of the body 102, 902 of the apparatus 100 and may allow for a larger and more comfortable range of hand motions when the apparatus 100 is worn (again, in comparison to tension-only links comprising rigid components).

Whether the link 106 is tension-and-compression or tension-only, the link 106 may be made to withstand an expected level of repeated loading for a suitable minimum number of loading cycles. The minimum number of loading cycles may be determined from a pre-determined design lifespan of the link 106 and the number of loading cycles expected to occur during that design lifespan. Repeated loading of the link 106 may occur as a result of involuntary and voluntary hand motions and as a result of the repeating movements of the link 106 in and out of the port 314. A flexible link 106 may result in fewer forces and in lower magnitudes of forces being experienced by at least some of the various components of the apparatus 100. Thus, a flexible link 106 may have (vis-à-vis tension-and-compression links) a longer lifespan and may increase the lifespan of the components of the apparatus 100 that are in contact with the flexible link 106 and may provide for quieter operation of the apparatus 100.

Referring still to FIGS. 3 to 5, the terminal 318 may be biased toward the first position 318a by any one or more of suitable means. For example, as shown, the terminal 318 may be biased with two springs 306 positioned in compression between a closure member 312 of the body 102 and the terminal 318. The springs 306 may exert a first force on the terminal 318 when the terminal 318 is in the first position 318a and a second force on the terminal 318 when the terminal 318 is in the second position 318b.

As shown, each of the springs 306 may be positioned over a spring guide 328. The spring guides 328 may have any suitable construction that may prevent or minimize buckling of the springs 306 which may occur but for the spring guides 328. In one example, each of the spring guides 328 may include a mating pair of rods 328a, 328b. Said pair of rods 328a, 328b may separate from each other as the terminal 318 moves from the second position 318b toward the first position 318a, and may enter into sliding contact with each other as the terminal 318 moves from the first position 318a toward the second position 318b.

The closure member 312 of the body 102 may seal the Non-Newtonian fluid in the internal space 316 while permitting the link 106 to move in and out of the port 314. The closure member 312 may be manufactured to be removably fixed to the body 102 (as shown), permanently fixed to the body 102, or integral with the body 102. The port 314 may include a sealing member 314a in sliding contact with the link 106 passing through the sealing member 314a and the port 314. In one example, the sealing member 314a may be an O-ring 314a positioned within the port 314.

The terminal 318 may divide the interior space 316 into a first 330 and second 332 sections. It will be appreciated that the volumes of the first 330 and second 332 sections may change as the terminal 318 moves between the first 318a and second 318b positions. The terminal 318 may comprise a resistance flow opening 302 connecting the first 330 and second 332 sections for bidirectional flow of the Non-Newtonian fluid, and a check valve 310 connecting the first 330 and second 332 sections for unidirectional flow of the Non-Newtonian fluid. The check valve 310 may be oriented to direct the unidirectional flow from the first 330 to the second 332 section of the interior space 316.

The check valve 310 may be any check valve suitable for the application. As shown, the check valve 310 may comprise a bypass flow opening 310a and a hinged flap 310b which may seal the bypass flow opening 310a when the terminal 318 moves from the first position 318a toward second the position 318b, thereby forcing the Non-Newtonian fluid to flow through the resistance flow opening 302 and thereby allowing for the desired damping forces to be generated at the terminal 318. The flap 310b may open the bypass flow opening 310a when the terminal 318 moves from the second position 318b toward first the position 318a, thereby allowing at least some of the Non-Newtonian fluid to bypass the resistance flow opening 302 by flowing through the bypass flow opening 310a and thereby reducing the forces required to move the terminal 318 in this direction.

In some cases, a diameter of the bypass flow opening 310a may be larger than a diameter of the resistance flow opening 302. Depending on the desired damping performance and dimensions of the body 102 and its components, the diameter of the bypass flow opening 310a may be made as large as possible and constrained only by a height of the terminal 318. The diameter of the resistance flow opening 302 may be selected depending on the particular Non-Newtonian fluid composition used with the body 102. It will be appreciated that the damping performance of the terminal 318 may be determined at least in part by the diameter of the resistance flow opening 302. The selection of the diameter of the resistance flow opening 302 may also depend on the height of the terminal 318 and may also depend on a width of the terminal 318.

In general, it may be desirable to size the openings 302 and 310a and other features of the body 102 to cooperatively provide a desired damping performance profile of the body 102. It may also be desirable to make the bypass flow opening 310a sufficiently large to allow the terminal 318 to move toward its biased first position 318a sufficiently quickly to reduce or substantially eliminate slack that may otherwise occur in the link 106 if the terminal 318 is too slow to move from the second 318b toward the first 318a position.

Making the diameter of the bypass flow opening 310a larger than the diameter of the resistance flow opening 302 may speed up said movement at least by providing the additional flow conduit formed by the bypass flow opening 310a. In embodiments where the bypass flow opening 310a is larger than the resistance flow opening 302, the larger diameter of the bypass flow opening 310a may allow for the Non-Newtonian fluid to flow through it with less resistance than when flowing through the resistance flow opening 302. Providing less resistance for the terminal 318 to move from the second position 318b toward the first position 318a may allow it to move in this direction quicker than from the first position 318b toward the second position 318a and may thereby reduce or substantially eliminate slack in the link 106 which may occur but for such features while and after a hand motion is made in a direction other than the direction in which the body 102, 902 may damp the involuntary hand motions.

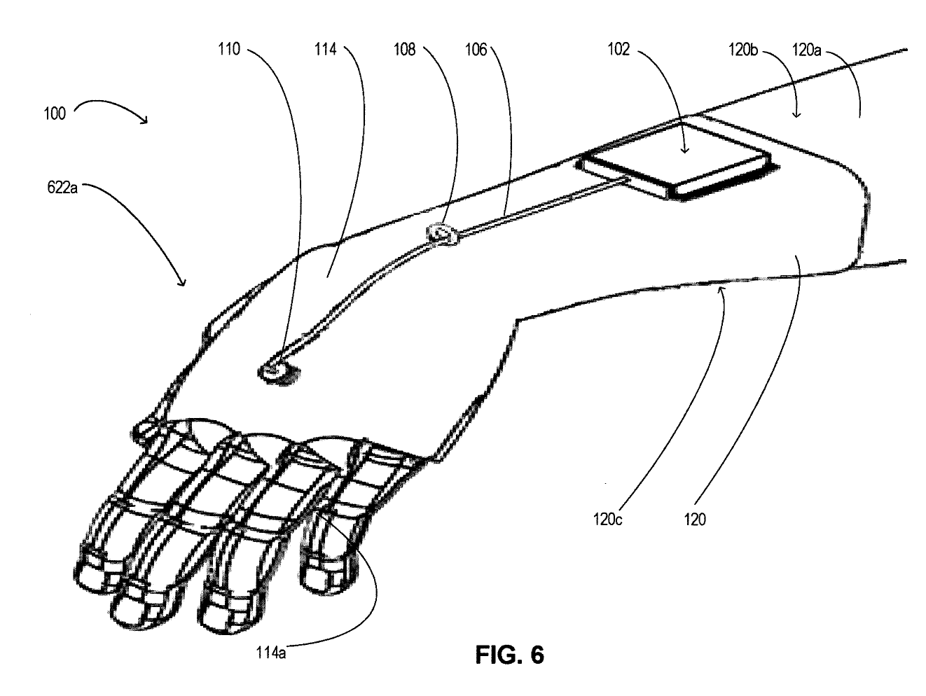

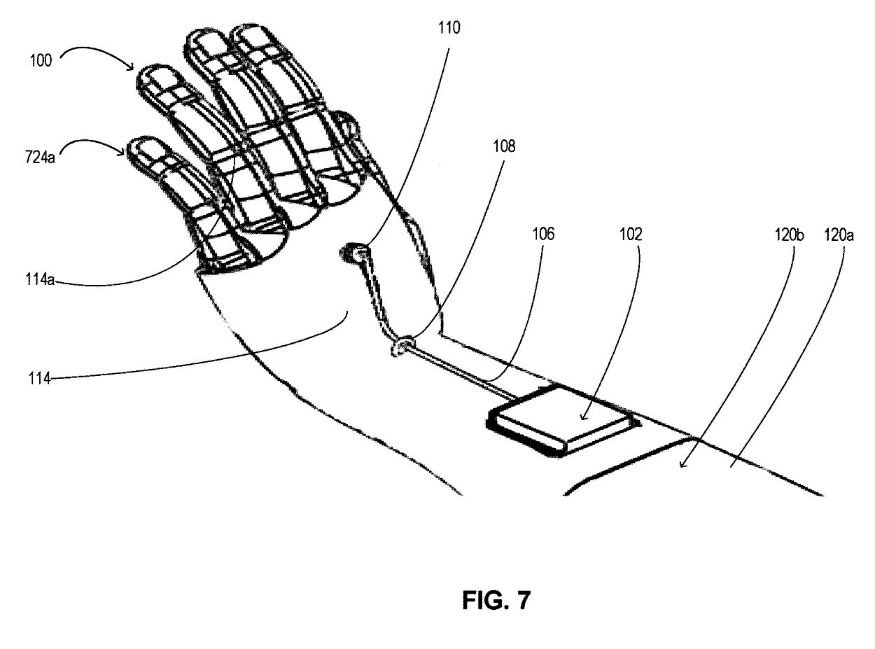

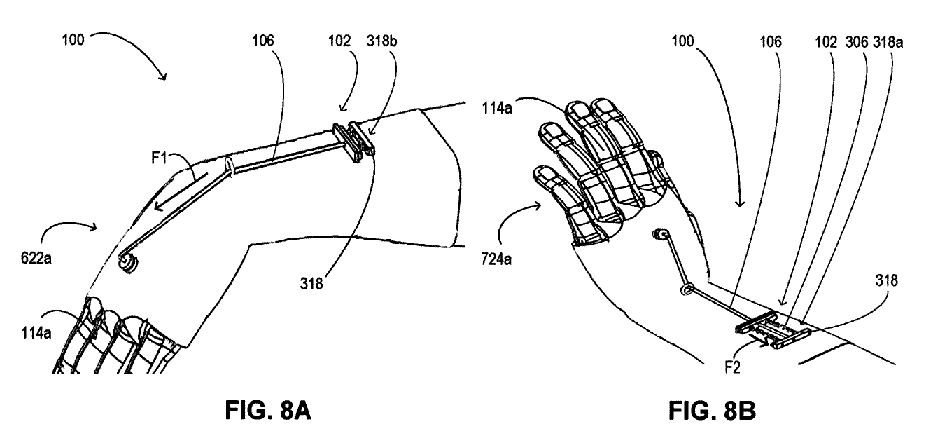

Reference is now made to FIGS. 6, 7, 8A, and 8B which show the apparatus 100 in two states: when the hand 114a is held in a fully-flexed position 622a, and when the hand 114a is held in a fully-extended position 724a, respectively. FIG. 8A corresponds to FIG. 6 and shows the body 102 of the apparatus 100 with the shell 320 hidden from this view to more clearly show the relationship of the features of this embodiment of the apparatus 100. Likewise, FIG. 8B corresponds to FIG. 7 and shows the body 102 with the shell 320 hidden from view for the same reasons. When the hand moves from the fully-extended position 724a toward the fully-flexed position 622a, it may exert a force F1 on the link 106. When the hand moves from the fully-flexed position 622a toward the fully-extended position 724a, the link 106 may (as described earlier) produce substantially no reactive forces in response to any forces that the hand while moving in this direction may apply at the connection 110 between the link 106 and the first portion 114, and therefore may transfer substantially no forces onto the terminal 318. Accordingly, in this direction, the terminal may move toward its biased position (first position 318a) predominantly as a result of at least one force F2 exerted on the terminal 318 by the means by which the terminal 318 may be biased toward the first position 318a.

In the example shown in FIGS. 6 to 8B, the hand 114a may move the terminal 318 from the first position 318a toward the second position 318b when the hand 114a moves from the fully-extended position 724a toward the fully-flexed position 622a. In other words, the terminal 318 may move from the first position 318a toward the second position 318b as a result of forces F1 that may be applied to terminal 318 by the hand 114a via the link 106. When the hand 114a moves from the fully-flexed position 622a toward the fully-extended position 724a, the terminal 318 may move from the second position 318b toward the first position 318a. However, the terminal 318 may move from the second position 318b toward the first position 318a as a result of forces F2 applied to the terminal 318 by the springs 306. In this embodiment, the terminal 318 may receive substantially no forces from the hand 114a while moving in the direction from the first 318a to the second 318b position.

In embodiments in which the body 102 is positioned on the dorsal side 120b of the hand 114a and the forearm 120a, the terminal 318 may be in the first 318a position when the hand 114a is fully-extended 724a and may be in the second position 318b when the hand is fully-flexed 622a. In embodiments in which the body 102 is positioned on the ventral side 120c of the hand 114a and the forearm 120a, the terminal 318 may be in the first position 318a when the hand 114a is fully-flexed 622a and may be in the second position 318b when the hand 114a is fully-extended 724a. In the latter embodiments, hand motions from the fully-extended position 724a toward the fully-flexed position 622a may move the terminal 318 from the second position 318b toward the first position 318a.

The internal space may have a first end-wall 434 and a second end-wall 436. The body 102 may be positioned relative to the second portion 120 and a length of the link 106 may be selected such that first position 318a is located at a first distance D1 away from the first end-wall 434 and the second position 318n is located at a second distance D2 away from the second end-wall 436. The first D1 and second D2 distances may be the same in some embodiments, and may be different in other embodiments. In this configuration, but for the tension in the link 106 and range of motion limitations of the hand 114a, the terminal 318 may move past the first position 318a toward the first end-wall 434 and past the second position 318b toward the second end-wall 436. This configuration may allow for the link 106 to remain in tension when the hand is in the fully-extended 724a and fully-flexed 622a positions. This configuration may be implemented for a body 102, 902 positioned on any one or both of the dorsal 120b and the ventral 120c sides for the hand 114a and the forearm 120a.

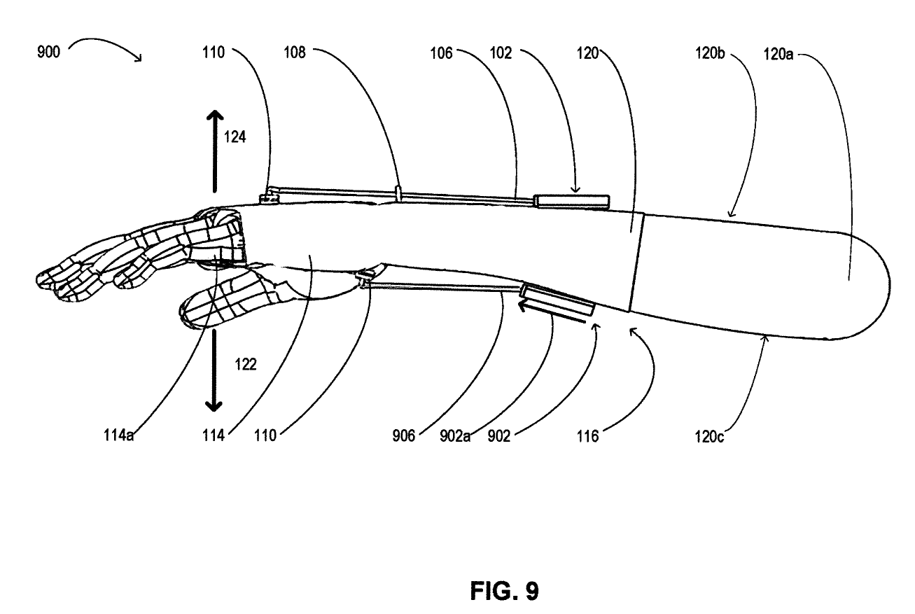

Reference is now made to FIG. 9. The apparatus 100, 200, 900 may comprise two bodies 102, 902. The bodies 102, 902 may be positioned relative to the first 114 and second 120 portions such that each of the bodies 102, 902 may dampen involuntary hand motions in one of first 122 and second 124 directions. Body 102 may be referred to as a first body 102. Body 902 may be referred to as a second body 902. The first body 102 may be positioned relative to the second portion 120 such that the first body 102 may be on the dorsal side 120b of the hand 114a and the forearm 120a when the first portion 114 is secured to the hand 114a and the second portion 120 is secured to the forearm 120a. The second body 902 may be positioned relative to the second portion 120 such that the second body 902 may be on the ventral side 120c of the hand 114a and the forearm 120a when the first portion 114 is secured to the hand 114a and the second portion 120 is secured to the forearm 120a.

The second body 902 may have similar features to the first body 102 and may be similarly secured to the second portion 120 and connected to the first portion 114 via the link 906 of the second body 902. When the first portion 114 is secured to the hand 114a and the second portion 120 is secured to the forearm 120a, the link 906 of the second body 902 may be in tension. In this example, the involuntary hand motions in the second direction 124 may move the terminal 318 of the second body 902 from the first position 318a of that terminal 318 toward the second position 318b of that terminal 318, in a direction shown in FIG. 9 with arrow 902a.

The next paragraphs describe possible performance profiles and methods of selecting Non-Newtonian fluid for the one or more bodies 102, 902 of the apparatus 100, 200, 900.

The Non-Newtonian fluid in each body 102, 902 may have a composition selected to achieve a damping performance profile of that body 102, 902 optimized for damping involuntary hand motions ranging in frequency from 2 to 12 hertz. In other words, varying loading applied by the involuntary hand motions to the link 106 of each body 102,902 may result in the terminal 318 of that body 102,902 generating varying damping forces in response to, proportional to, and optimized for the varying loading.

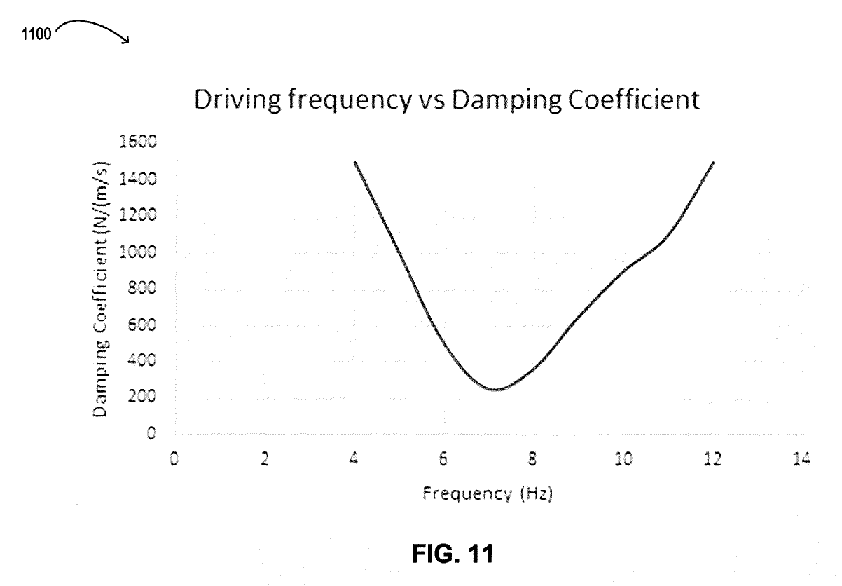

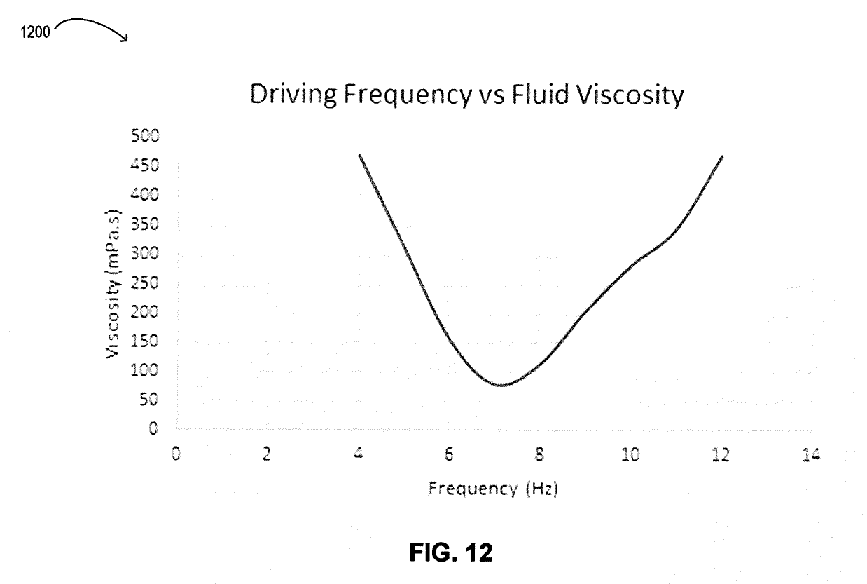

In a preferred embodiment, the features of each body 102, 902 and the composition of the Non-Newtonian fluid of that body may be selected to result in the terminal 318 of that body 102, 902 producing damping forces according to a damping performance profile 1100 as shown in FIG. 11. This damping performance profile 1100 is represented as a damping coefficient of the terminal 318 as a function of frequency of movements of the terminal 318 (“driving frequency”). In one example, this damping performance profile 1100 may be achieved by using Non-Newtonian fluid selected to exhibit viscosity changes according to a viscosity performance profile 1200 as shown in FIG. 12, which changes may result in response to the driving frequency received by the fluid from the terminal 318.

In general, damping performance profiles 1100 of each body 102, 902 of the apparatus 100, 200, 900 may be optimized for a particular user's involuntary hand motions or for a range of different types of involuntary hand motions that may be experienced by different users.

Viscosity and other properties of Non-Newtonian fluid may be a function of the combination of size(s), shape(s), and concentration(s) of the particles of that Non-Newtonian fluid and the carrier fluid(s) of that Non-Newtonian fluid. Non-Newtonian fluid may be selected for a given body 102, 902 by using any suitable combination of known selection and testing methods. In particular, Non-Newtonian fluid may be selected by iteratively selecting and testing particular combinations of features of the given body 102, 902 in combination with particular compositions of Non-Newtonian fluid until a desired damping performance curve of that body 102, 902 is achieved. Suitable types of fluid and its various components may depend on the features and materials chosen for the given body 102, 902 and will be ascertainable by a person skilled in the art without undue experimentation.

A suitable testing method for the given body 102, 902 may include using a damper test rig equipped with: an electromagnetic shaker to simulate varying involuntary hand motions and apply them to the link 106 of that body 102, 902, a load cell to measure the reaction forces generated by that body 102, 902, an accelerometer to measure the frequency and amplitude of the resulting motion of the terminal 318 of that body 102, 902, and a data acquisition computer to collect all resulting information. Another suitable testing method may include the use of suitable Viscometer machine, such as those used in rheology to test the performance of fluids under different frequencies and amplitudes of loading and under different temperatures.

In embodiments in which the apparatus 100, 200, 900 includes two bodies 102, 902, each body 102, 902 and its respective Non-Newtonian fluid may be selected at least as described above to result in a damping performance profile specific to each body 102, 902. In some embodiments, the damping performance profiles may differ, depending on the dynamics of the involuntary hand motions that each body 102, 902 may be optimized to dampen. In other embodiments, the damping performance profiles may be the same.

The damping performance profile(s) may be optimized for the particular involuntary hand motions experienced by a particular user. The damping performance profile(s) may also be optimized for damping involuntary hand motions that occur within a given range of frequencies. In a particular example, the damping performance profile(s) may be optimized for involuntary hand motions occurring at a frequency falling within the range of 2 hz to 12 hz. A majority of the most common types of involuntary hand motions may occur in the range of 2 hz to 12 hz.

The next paragraphs describe suitable materials, methods of manufacture, and a preferred embodiment of the apparatus 100.

In some embodiments, the first 114 and second 120 portions may be portions of a glove 116. In these embodiments, the first 114 and second 120 portions may be manufactured to be secured removably or permanently to the glove 116. The terms removably or permanently may be taken to have similar meanings as described earlier for the same terms in relation to the body 102. In some embodiments, including embodiments where the first 114 and second 120 portions are portions of a glove 116, the first 114 and second 120 portions may be manufactured integral to or separate from each other.

The apparatus 100, 200, 900 may be manufactured to have a sufficiently small geometry relative to the hand 114a and the forearm 120a for at least a portion of the apparatus 100, 200, 900 to be concealable under a garment worn by a user of the apparatus 100, 200, 900. In some cases, the body 102, 902 of the apparatus 100, 200, 900 may be manufactured to be permanently or selectively concealed in the first 114 and second 120 portions. In embodiments where the apparatus 100, 200, 900 includes a glove 116, the body 102, 902 may manufactured to be permanently or selectively concealed in the glove 116. In other embodiments, a stand-alone glove (not shown) may be provided separate from and not meant to be secured to the first 114 and second 120 portions. The stand-alone glove may be configured to conceal at least a part of each of the first 114 and second 120 portions and the body 102, 902.

In some embodiments, the 114 and second 120 portions may include collars (not shown) or belt-type securing portions (not shown). In other embodiments, the 114 and second 120 portions may be manufactured as stand-alone portions having an adhesive layer (not shown) on at least one side of said portions 114, 120 for securing the portions 114, 120 to the hand 114a and the forearm 120a, respectively.

The apparatus 100, 200, 900 may be manufactured using any combination of known materials and manufacturing and assembly methods suitable for each particular embodiment of the apparatus 100, 200, 900. For example, the glove 116 may be made from any materials which will make the glove 116 sufficiently elastic to allow for comfortable hand motions and at the same time will compress the hand 114a and the forearm 120a sufficiently to provide for acceptable levels of slack and of movements of the components of the apparatus 100, 200, 900. Levels of slack and of movements of the components may be unacceptable where, for example, the glove 116 materials are chosen such that the forces resulting from involuntary hand motions will deform the glove 116 instead of moving the terminal 318 of the body 102, 902. Desired levels of slack and of movements of the components may be dictated by particular demands for comfort of particular users of the apparatus 100, 200, 900.

Materials for the glove 116 may be, for example, selected to include any one or a combination of materials chosen from the following group: Spandex, Cotton, Coolmax, Thermoplastics, Polyspandex, Nylon, Bamboo, Neoprene, Vinyl, Terry foam, and contour foam.

The shell 320, the closure member 312, the terminal 318, the check valve 310, the sealing member 314a, and the springs 306 of the body 102, 902 may each be made from any known suitable material such as metal, rubber, plastic, or other materials, so long as the materials in combination provide for the functionality described in this document. In some cases, the components of the body 102, 902 may be individually or integrally 3D-printed, cast, or injection molded. Whether the springs 306 can be 3D printed may change with the evolution of 3D printing technology.

The body 102, 902 may be secured to the second portion 120 either removably or permanently, using any suitable securement. Examples of suitable securements may include any one or a combination of sewing, gluing, and mechanical mechanisms for removable securement such as pairs of mating securement members (not shown). In some embodiments, insulation (not shown) may be attached around the body 102, 902. In embodiments where the apparatus 100, 200, 900 includes a glove 116, the insulation may be part of the glove 116. The insulation may slow down the rates at which the various components of the apparatus 100, 200, 900 may experience temperature changes in response to varying ambient temperature conditions. Slower rates of temperature changes may provide for a more stable operation of the apparatus.

The link(s) 106, 906 may be made using any suitable materials that may include any one or a combination of: cable, string, and flexible plastic. Connectors 1000 (if any) corresponding to the link(s) 106, 906 may likewise be made from any suitable material(s) and may be selected from suitable off-the-shelf items such as riveted buttons 1002.

In a variation of a second embodiment, the apparatus may have two bodies 102, 902 as shown in FIG. 9 and as described above. The two bodies 102, 902, except for the links 106, may be 3D-printed using a suitable plastic. The check valves 310 may be 3D-printed integrally with their respective terminal 318. Each terminal 318 may be biased using two springs 306, which may be helical metal compression springs each having a spring constant in the range of 0.1 to 0.5 N/mm. Each of the two links 106 may be a weaved-metal cable with a flexible plastic sheath having a smooth outer surface. The rubber sheath may have an external diameter of one tenth of an inch. Each of the two sealing member 314a may be a rubber o-ring 314a frictionally secured within the port 314 and contacting a circumference of the flexible plastic sheath of the link 106 at an internal circumference of the o-ring 314a and allowing the sheath (and the link 106) to move with minimal friction relative to the o-ring 314a while keeping Non-Newtonian fluid sealed in each of the two internal spaces 316.

The internal spaces 316 and the terminals 318 of the two bodies 102, 902 may be rectangular and may have cooperatively matching dimensions. Each of the two bodies 102, 902 may have a height 326a of one-half of an inch and a width 326b and length 326c of two inches. End each of the terminals 318 may be slidably fitted into its respective internal space 316. The diameter of the resistance flow opening 302 may be one tenth of an inch and the diameter of the bypass flow opening 310a maybe two tenths of an inch.

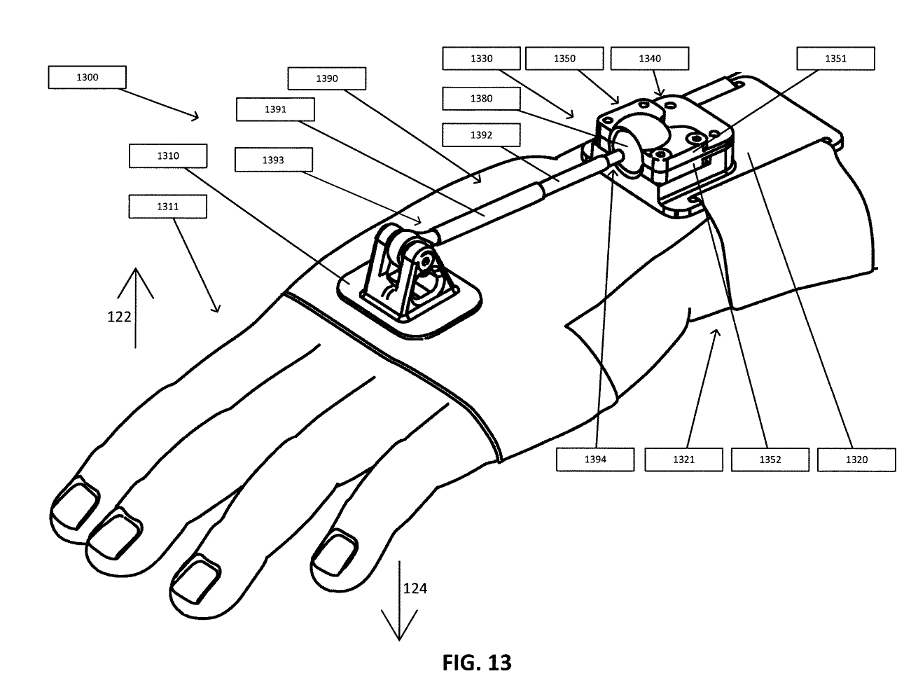

Reference is now made to FIG. 13 and a variation of a third embodiment. The involuntary hand motions may occur in at least a first and second direction 122, 124, the first direction 122 being opposite the second direction 124. FIG. 13 shows an example of an apparatus 1300 for damping involuntary hand motions occurring in at least a first 122 and second 124 directions, the first direction 122 being opposite the second direction 124. As shown, the apparatus 1300 may comprise a first portion 1310 securable to a hand 1311 and a second portion 1320 securable to a forearm 1321 and movable relative to the first portion 1310. The apparatus 1300 may further comprise a body 1330 secured to the second portion 1320. The body 1330 may dampen the involuntary hand motions in one of the at least first 122 and second 124 directions. The apparatus 1300 is designed to dampen the involuntary hand motions occurring in at least first 122 and second 124 directions, preferably without restricting voluntary hand motions in any direction.

In some embodiments, the body 1330 may be permanently secured to the second portion 1320. In other embodiments, the body 1330 may be removably secured to the second portion 1320. A permanent securement may be a securement designed to not be altered by a user of the apparatus 1300 after manufacturing and assembly of the apparatus 1300. A removable securement may include mating portions, at least one on each of the second portion 1320 and the body 1330. The mating portions may hold the body 1330 in place for operation of the apparatus 1300 and may allow for the user or a technician to detach the body 1330 from the second portion 1320 for maintenance or replacement. In some embodiments, the removable securement may be configured to allow for adjusting the position of the body 1330 relative to the second portion 1320. Any suitable combination of known methods and components may be used to implement any one or a combination of the permanent and removable securements.

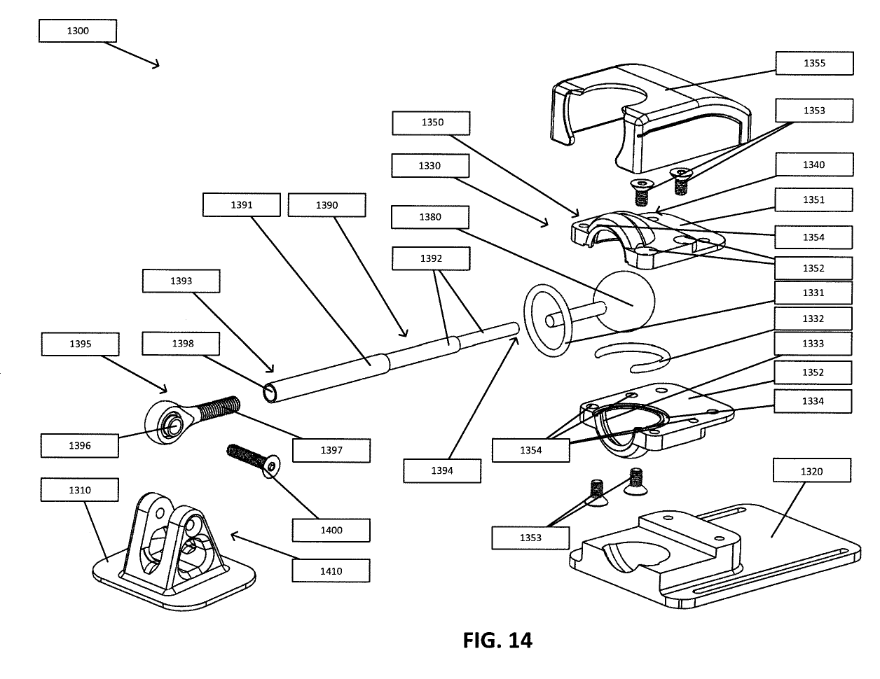

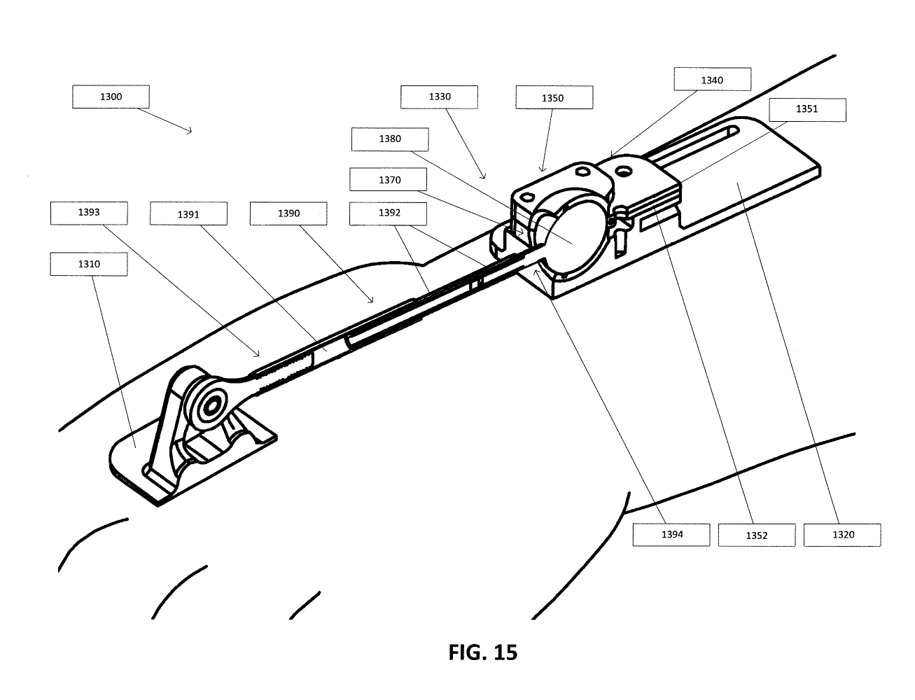

Reference is now made to FIGS. 14 and 15. The body 1330 may comprise a casing 1350, which may comprise an exterior surface 1340 and may be made of two halves 1351 and 1352, defining a spherical interior space and having a port 1370 extending from the spherical interior space to the exterior surface 1340. The two halves 1351 and 1352 may be attached by means of threaded inserts 1353 inserted into pre-formed threaded openings 1354. The body 1330 further comprises a connector comprising a spherical terminal 1380 inside the casing 1350, filling the casing save for a gap filled with a shear thickening fluid (not shown). The connector may also comprise a link 1390 passing through the port 1370 and connecting the spherical terminal 1380 to the first portion 1310. A protective housing 1355 may be provided over at least a portion of the body 1330 to protect the body from damage or tampering.

As shown in FIGS. 14 and 15, in a first variation, the link 1390 may be comprised of one main stage 1391 and at least one additional stage 1392, to form a telescoping cylinder having first 1393 and second ends 1394. This telescoping cylinder would allow the involuntary hand motions occurring in at least a first 122 and second 124 directions to occur without interference from the link 1390 and to be passed on to the spherical terminal 1380. The link may further comprise a connector attachment 1395 attached to first end 1393. This connector attachment 1395 may have an aperture 1396 and may be attached to the telescoping cylinder by means of first threading 1397 on the end of the attachment 1395 farthest from the aperture 1396. This first threading may correspond to a threaded hole 1398 in the telescoping cylinder.

As shown in FIGS. 14 and 15, the link 1390 may be rigidly fixed to the spherical terminal 1380. Any suitable combination of known methods and components may be used to implement this fixation. In one variation the link 1390 may comprise second threading (not shown) on the second end 1394 of the telescoping cylinder. This second threading may correspond to a threaded hole (not shown) in the spherical terminal 1380, allowing the link 1390 to be attached rigidly to the spherical terminal 1380.

Continuing to refer to FIGS. 14 and 15, the link 1390 may connect the spherical terminal 1380 to the first portion 1310 via any suitable connection. The link 1390 may include an aperture 1396 on the end opposite the spherical terminal 1380 through which a rod 1400 may pass, such that the link 1390 may pivot freely around the rod 1400. The rod 1400 may form a part of a raised base 1410 attached to the first portion 1310. Connections between the link 1390 and the first portion 1310 may also be made by any other suitable means. For example by tying or permanently attaching link 1390 to first portion 1310.

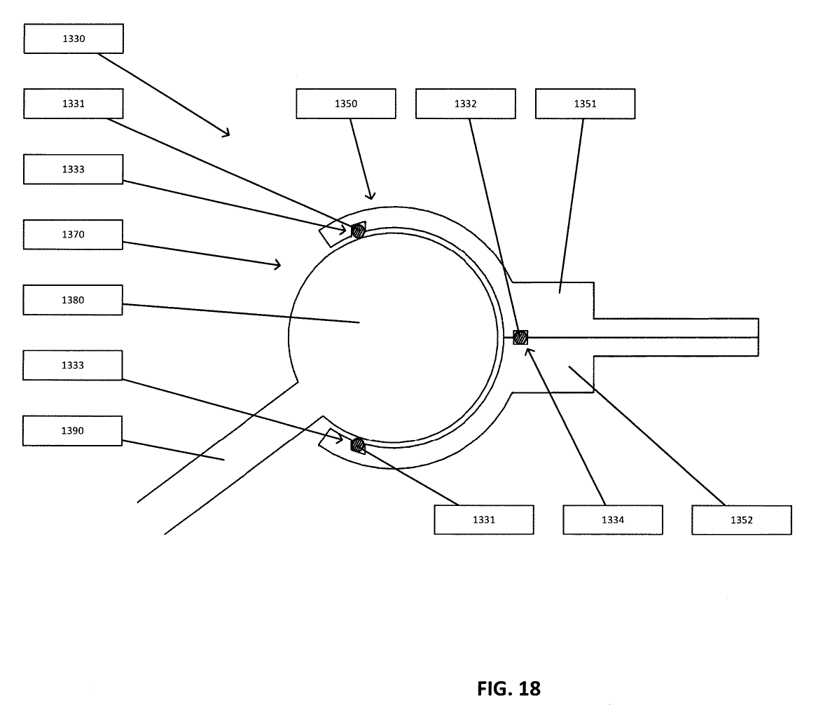

As shown in FIGS. 14 and 18, the body 1330 may further comprise elastic o-ring 1331 and partial o-ring 1332. This o-ring 1331 and partial o-ring 1332 would be positioned in troughs 1333 and 1334, respectively, and frictionally held in place. The partial o-ring 1332 would be in contact with both halves of the casing 1350, the o-ring 1331 would be in contact with the spherical terminal 1380 and both halves of the casing 1350. This o-ring 1331 and partial o-ring 1332 would assist in blocking the shear thickening fluid (not shown) positioned in the gap between the spherical terminal 1380 and the casing 1350 from leaking out of the gap or between the two halves 1351 and 1352.

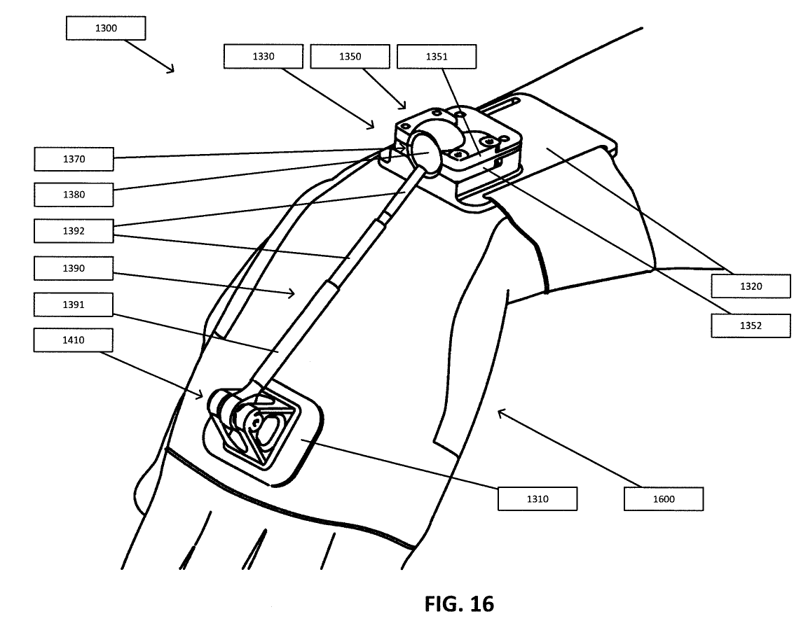

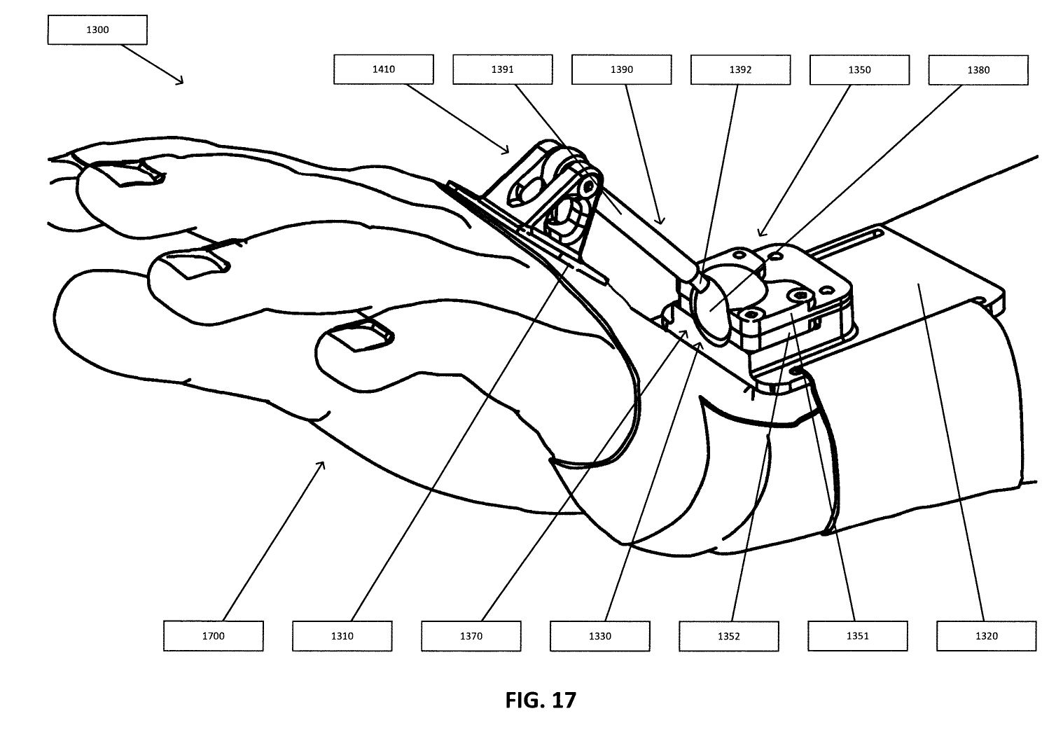

Reference is now made to FIGS. 16 and 17 which show the apparatus 1300 in two states: when the hand 1311 is held in a fully-flexed position 1600, and when the hand 1311 is held in a fully-extended position 1700, respectively. When the hand moves from the fully-extended position 1700 toward the fully-flexed position 1600, it may cause the spherical terminal 1380 to rotate within the casing 1350. When the hand moves from the fully-flexed position 1600 toward the fully-extended position 1700, it may also cause rotation of the spherical terminal 1380 within the casing 1350. This rotation may cause sheer friction between the spherical terminal 1380 and the shear thickening fluid (not shown).

The next paragraphs describe possible performance profiles and methods of selecting the shear thickening fluid for body 1330 of the apparatus 1300.

The shear thickening fluid in body 1330 may have a composition selected to achieve a damping performance profile of that body 1330 optimized for damping involuntary hand motions ranging in frequency from 2 to 12 hertz. In other words, varying loading applied by the involuntary hand motions to the link 1390 of body 1330 may result in the spherical terminal 1380 of body 1330 generating varying damping forces in response to, proportional to, and optimized for the varying loading.

The following four fluid mixtures are provided as non-limiting examples of appropriate shear thickening fluids. These mixtures are comprised of various proportions of fumed nanosilica which may be obtained from Cabot Corporation, polypropylene glycol with a viscosity of 115 cSt which may be obtained from Sigma Aldrich, and high-viscosity silicone oil with a viscosity of 2500 Pa-s which may be obtained from Clearco Products. A first example mixture is comprised of 28% fumed nanosilica, 41% polypropylene glycol and 31% high-viscosity silicone oil, by weight. A second example mixture is comprised of polypropylene glycol containing 30% fumed nanosilica by weight, cumulatively combined in equal weight with high-viscosity silicone oil. A third example mixture is comprised of an equal weight portion of polypropylene glycol and high-viscosity silicone oil combined with fumed nanosilica particles which comprise 25% of the total weight of the fluid. A fourth example mixture is comprised of a mixture of fumed nanosilica particles in low-viscosity hydroxyl-terminated silicone oil, wherein the mixture contains nanosilica in greater than 30 parts per hundred.

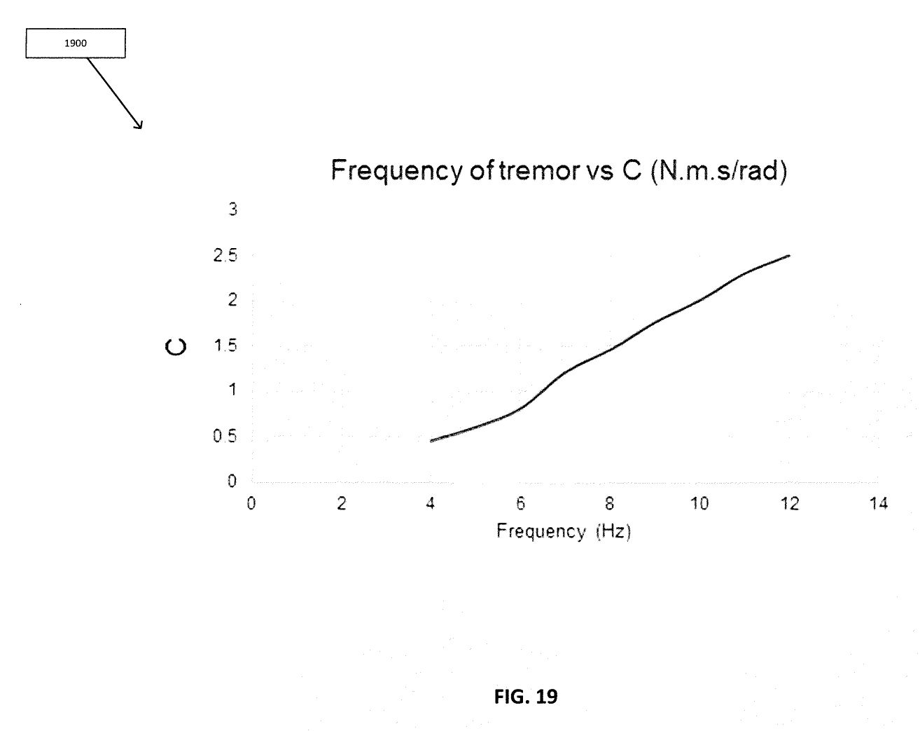

In a preferred embodiment, the features of body 1330 and the composition of the shear thickening fluid of that body may be selected to result in the spherical terminal 1380 of that body 1330 producing damping forces according to a damping performance profile 1900 as shown in FIG. 19. This damping performance profile 1900 is represented as a damping coefficient of the terminal 1380 as a function of frequency of movements of the spherical terminal 1380 (“driving frequency”). In one example, this damping performance profile 1900 may be achieved by using shear thickening fluid selected to increase in viscosity in response to increased shear rates of the spherical terminal 1380.

In general, damping performance profiles 1900 of body 1330 of the apparatus 1300 may be optimized for a particular user's involuntary hand motions or for a range of different types of involuntary hand motions that may be experienced by different users.

Viscosity and other properties of shear thickening fluid may be a function of the combination of size(s), shape(s), and concentration(s) of the particles of that shear thickening fluid and the carrier fluid(s) of that shear thickening fluid. The shear thickening fluid may be selected for a given body 1330 by using any suitable combination of known selection and testing methods. In particular, shear thickening fluid may be selected by iteratively selecting and testing particular combinations of features of the given body 1330 in combination with particular compositions of shear thickening fluid until a desired damping performance curve of that body 1330 is achieved. Suitable types of fluid and its various components may depend on the features and materials chosen for the given body 1330 and will be ascertainable by a person skilled in the art without undue experimentation.

A suitable testing method for the given body 1330 may include using a damper test rig equipped with: an electromagnetic shaker to simulate varying involuntary hand motions and apply them to the link 1390 of that body 1330, a load cell to measure the reaction forces generated by that body 1300, an accelerometer to measure the frequency and amplitude of the resulting motion of the spherical terminal 1380 of that body 1330, and a data acquisition computer to collect all resulting information. Another suitable testing method may include the use of suitable Viscometer machine, such as those used in rheology to test the performance of fluids under different frequencies and amplitudes of loading and under different temperatures.

The damping performance profile(s) may be optimized for the particular involuntary hand motions experienced by a particular user. The damping performance profile(s) may also be optimized for damping involuntary hand motions that occur within a given range of frequencies. In a particular example, the damping performance profile(s) may be optimized for involuntary hand motions occurring at a frequency falling within the range of 2 hz to 12 hz. A majority of the most common types of involuntary hand motions may occur in the range of 2 hz to 12 hz.

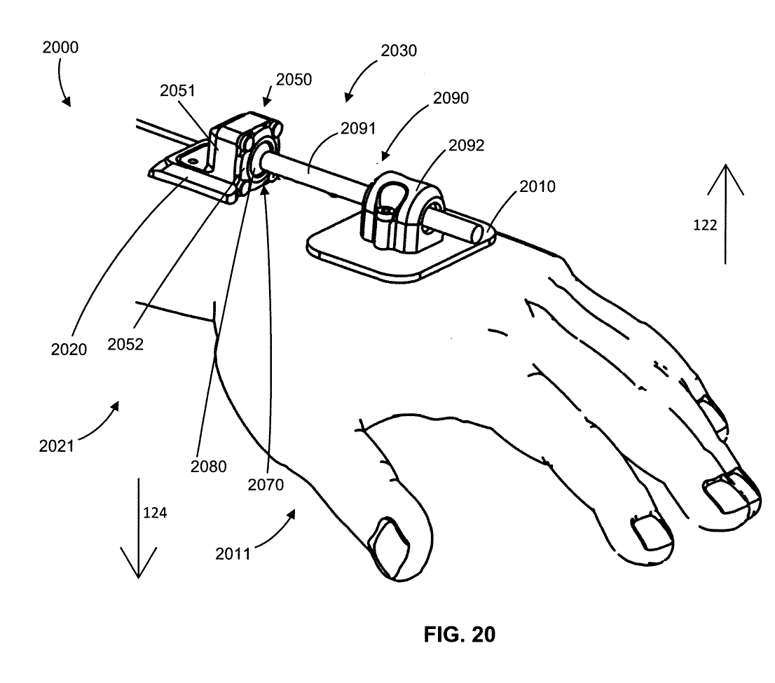

As shown in FIGS. 20 to 25, in a second variation of a third embodiment, the link 2090 may be comprised of a shaft 2091 and a linear ball bearing 2092, rather than a telescoping cylinder.

Reference is now made to FIG. 20. The involuntary hand motions may occur in at least a first and second direction 122, 124, the first direction 122 being opposite the second direction 124. FIG. 20 shows an example of an apparatus 2000 for damping involuntary hand motions occurring in at least a first 122 and second 124 directions, the first direction 122 being opposite the second direction 124. As shown, the apparatus 2000 may comprise a first portion 2010 securable to a hand 2011 and a second portion 2020 securable to a forearm 2021 and movable relative to the first portion 2010. The apparatus 2000 may further comprise a body 2030 secured to the second portion 2020. The body 2030 may dampen the involuntary hand motions in one of the at least first 122 and second 124 directions. The apparatus 2000 is designed to dampen the involuntary hand motions occurring in first 122 and second 124 directions, preferably without restricting voluntary hand motions in any direction.

In some embodiments, the body 2030 may be permanently secured to the second portion 2020. In other embodiments, the body 2030 may be removably secured to the second portion 2020. A permanent securement may be a securement designed to not be altered by a user of the apparatus 2000 after manufacturing and assembly of the apparatus 2000. A removable securement may include mating portions, at least one on each of the second portion 2020 and the body 2030. The mating portions may hold the body 2030 in place for operation of the apparatus 2000 and may allow for the user or a technician to detach the body 2030 from the second portion 2020 for maintenance or replacement. In some embodiments, the removable securement may be configured to allow for adjusting the position of the body 2030 relative to the second portion 2020. Any suitable combination of known methods and components may be used to implement any one or a combination of the permanent and removable securements.

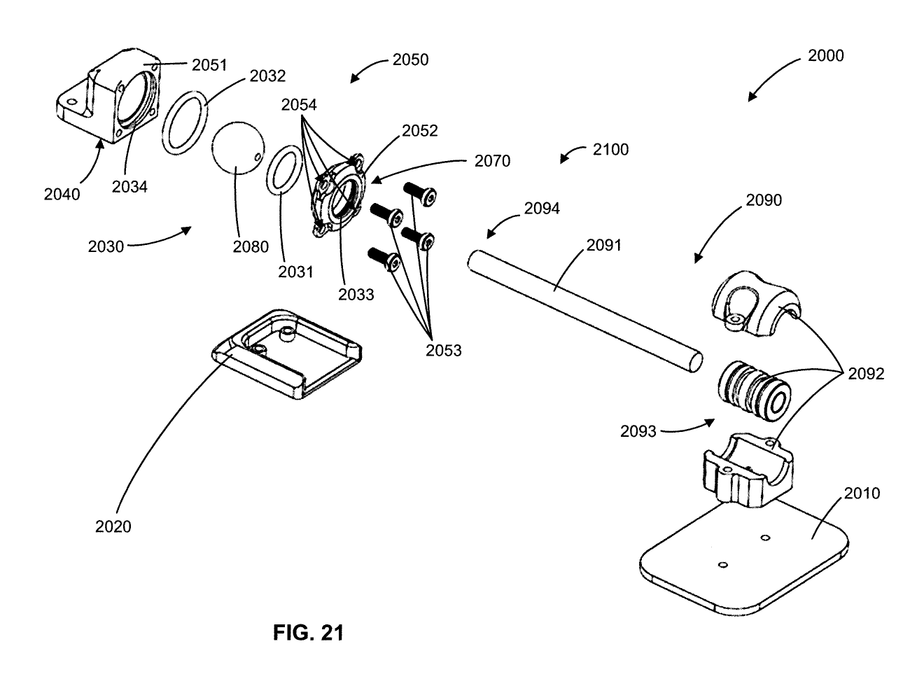

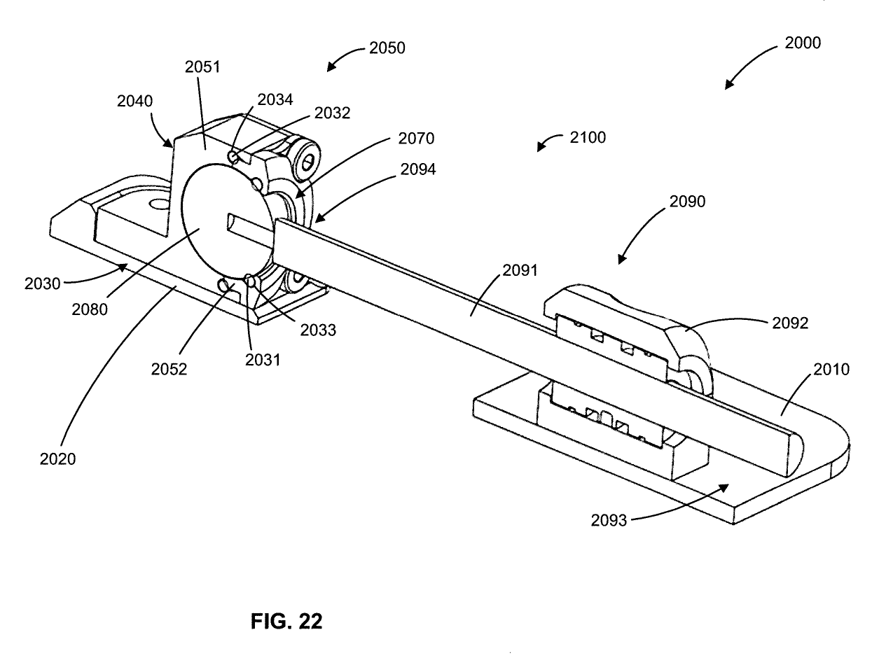

Reference is now made to FIGS. 21 and 22. The body 2030 may comprise a casing 2050, which may comprise an exterior surface 2040 and which may be made of two portions 2051 and 2052, defining a spherical interior space and having a port 2070 extending from the spherical interior space to the exterior surface 2040. The two portions 2051 and 2052 may be attached to one another by means of threaded inserts 2053 inserted into pre-formed threaded openings 2054. The body 2030 further comprises a connector 2100, which may comprise a spherical terminal 2080 inside the casing 2050, filling the casing save for a gap filled with a shear thickening fluid (not shown). The connector 2100 may also comprise a link 2090 passing through the port 2070 and connecting the spherical terminal 2080 to the first portion 2010. A protective housing (not shown) may be provided over at least a portion of the body 2030 to protect the body from damage or tampering.

As shown in FIGS. 21 and 22, in a second variation, the link 2090 may be comprised of a shaft 2091 and a linear bearing 2092, which may be a linear ball bearing; to form a linear bearing link having first 2093 and second ends 2094. This link would allow the involuntary hand motions occurring in at least a first 122 and second 124 directions to occur without interference from the link 2090 and to be passed on to the spherical terminal 2080. Linear bearings and linear ball bearings are commercially available, and may be made of various materials, such as Ceramic or polytetrafluoroethylene (PTFE). Linear bearings and linear ball bearings may provide the benefit of very low friction movement, and may reduce the possibility of a jamming of components of the link. As the user's hand moves, the shaft 2091 moves within the linear bearing 2092, compensating for any changes in distance between the spherical terminal 2080 and the linear bearing 2092. A link comprising a linear bearing may also smooth force transmission to the hand. A link comprising a linear bearing may enable radial and ulnar deviation movement of the hand, as the shaft 2091 may not be forced to move relative to the linear bearing 2092 as it may be able to freely rotate within the linear bearing 2092.

As shown in FIGS. 21 and 22, the link 2090 may be rigidly fixed to the spherical terminal. Any suitable combination of known methods and components may be used to implement this fixation. In one variation the link 2090 may comprise threading (not shown) on the second end 2094 of the shaft 2091. This second threading may correspond to a threaded hole (not shown) in the spherical terminal 2080, allowing the link 2090 to be attached rigidly to the terminal 2080.

Continuing to refer to FIGS. 21 and 22, the link 2090 may connect the spherical terminal 2080 to the first portion 2010, and the link may be fastened to the first portion 2010 via any suitable connection, including a rigid connection or a pivotal connection permitting the link 2090 to move relative to the first portion 2010.

As shown in FIG. 21, the body 2030 may further comprise elastic o-rings 2031 and 2032. These o-rings 2031 and 2032 would be positioned in troughs 2033 and 2034, respectively, and frictionally held in place. O-ring 2032 would be in contact with both portions of the casing 2050, and o-ring 2031 would be in contact with the spherical terminal 2080 and both portions of the casing 2050. These o-rings 2031 and 2032 would block the shear thickening fluid (not shown) positioned in the gap between the spherical terminal 2080 and the casing 2050 from leaking out of the gap.

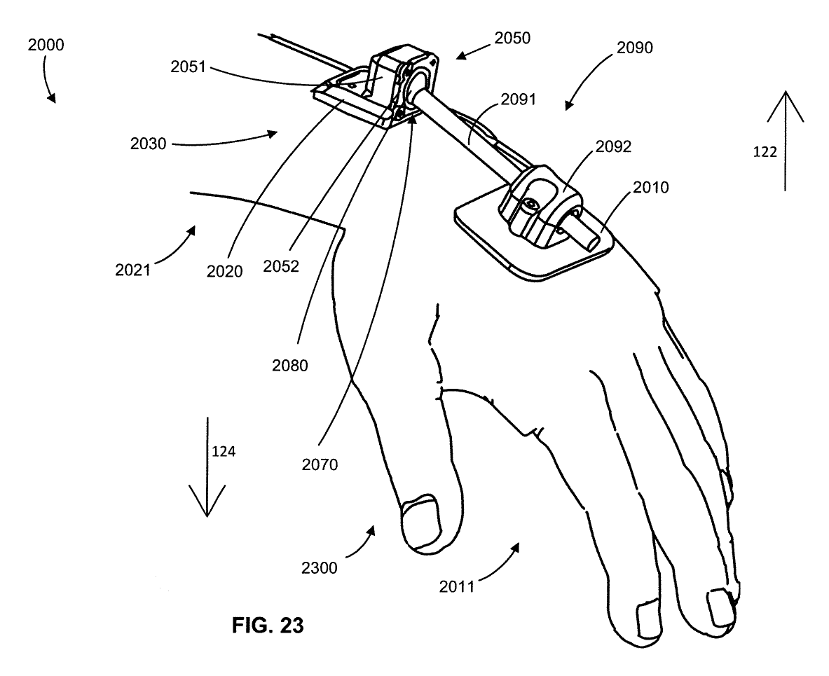

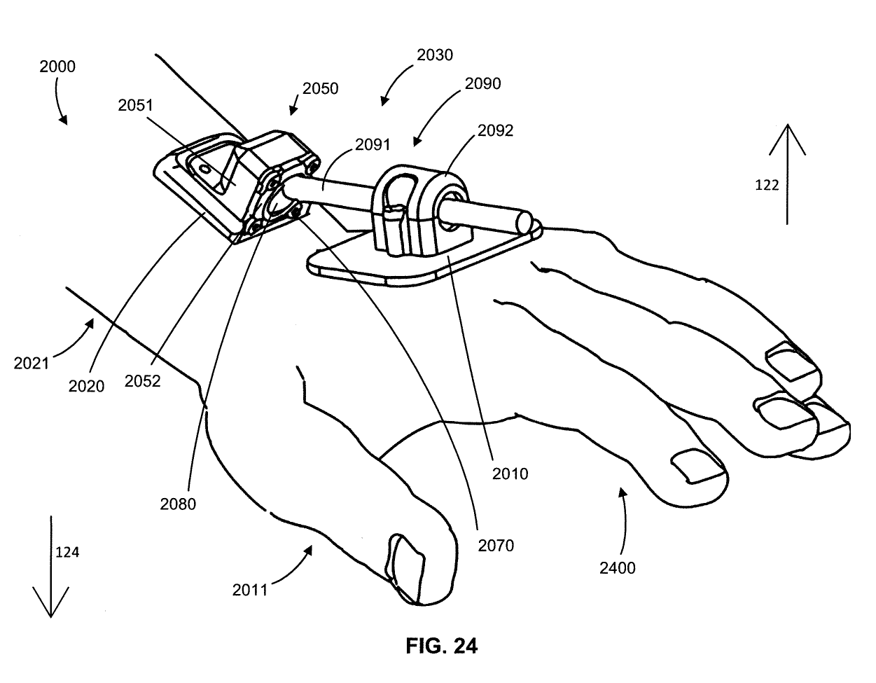

Reference is now made to FIGS. 23 and 24 which show the apparatus 2000 in two states: when the hand 2011 is held in a fully-flexed position 2300, and when the hand 2011 is held in a fully-extended position 2400, respectively. When the hand moves from the fully-extended position 2400 toward the fully-flexed position 2300, it may cause the spherical terminal 2080 to rotate within the casing 2050. When the hand moves from a fully-flexed position 2300 toward the fully-extended position 2400, it may also cause rotation of the spherical terminal 2080 within the casing 2050. This rotation may cause sheer friction between the spherical terminal 2080 and the shear thickening fluid (not shown).

The next paragraphs describe possible performance profiles and methods of selecting the shear thickening fluid for body 2030 of the apparatus 2000.

The shear thickening fluid in body 2030 may have a composition selected to achieve a damping performance profile of that body 2030 optimized for damping involuntary hand motions ranging in frequency from 2 to 12 hertz. In other words, varying loading applied by the involuntary hand motions to the link 2090 of body 2030 may result in the spherical terminal 2080 of body 2030 generating varying damping forces in response to, proportional to, and optimized for the varying loading.

As described above, the following four fluid mixtures are provided as non-limiting examples of appropriate shear thickening fluids. These mixtures are comprised of various proportions of fumed nanosilica which may be obtained from Cabot Corporation, polypropylene glycol with a viscosity of 115 cSt which may be obtained from Sigma Aldrich, and high-viscosity silicone oil with a viscosity of 2500 Pa-s which may be obtained from Clearco Products. A first example mixture is comprised of 28% fumed nanosilica, 41% polypropylene glycol and 31% high-viscosity silicone oil, by weight. A second example mixture is comprised of polypropylene glycol containing 30% fumed nanosilica by weight, cumulatively combined in equal weight with high-viscosity silicone oil. A third example mixture is comprised of an equal weight portion of polypropylene glycol and high-viscosity silicone oil combined with fumed nanosilica particles which comprise 25% of the total weight of the fluid. A fourth example mixture is comprised of a mixture of fumed nanosilica particles in low-viscosity hydroxyl-terminated silicone oil, wherein the mixture contains nanosilica in greater than 30 parts per hundred.

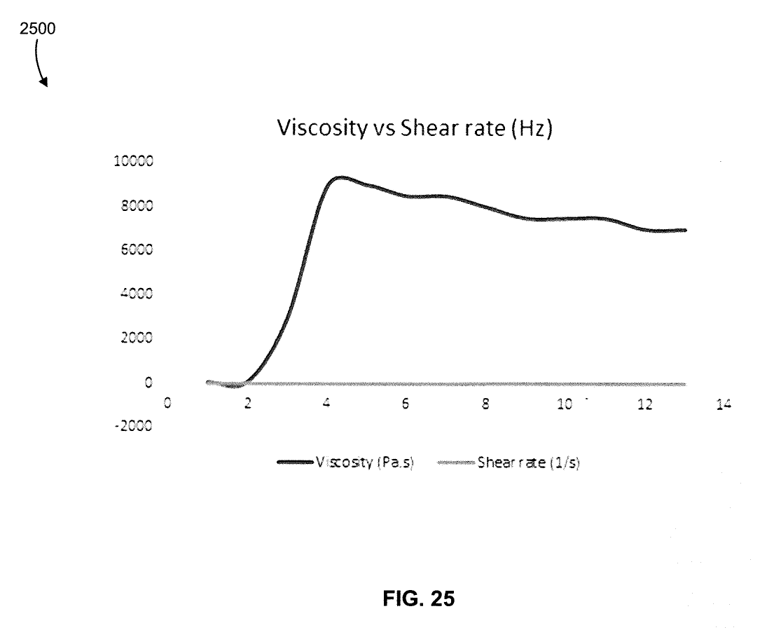

In a preferred embodiment, the features of body 2030 and the composition of the shear thickening fluid of that body may be selected to result in the spherical terminal 2080 of that body 2030 producing damping forces according to a damping performance profile 2500 as shown in FIG. 25. This damping performance profile 2500 is represented as a damping coefficient of the spherical terminal 2080 as a function of frequency of movements of the spherical terminal 2080 (“driving frequency”). In one example, this damping performance profile 2500 may be achieved by using shear thickening fluid selected to increase in viscosity in response to increased shear rates of the spherical terminal 2080.

In general, damping performance profiles 2500 of body 2030 of the apparatus 2000 may be optimized for a particular user's involuntary hand motions or for a range of different types of involuntary hand motions that may be experienced by different users.

Viscosity and other properties of shear thickening fluid may be a function of the combination of size(s), shape(s), and concentration(s) of the particles of that shear thickening fluid and the carrier fluid(s) of that shear thickening fluid. The shear thickening fluid may be selected for a given body 2030 by using any suitable combination of known selection and testing methods. In particular, shear thickening fluid may be selected by iteratively selecting and testing particular combinations of features of the given body 2030 in combination with particular compositions of shear thickening fluid until a desired damping performance curve of that body 2030 is achieved. Suitable types of fluid and its various components may depend on the features and materials chosen for the given body 2030 and will be ascertainable by a person skilled in the art without undue experimentation.

A suitable testing method for the given body 2030 may include using a damper test rig equipped with an electromagnetic shaker to simulate varying involuntary hand motions and apply them to the link 2090 of that body 2030, a load cell to measure the reaction forces generated by that apparatus 2000, an accelerometer to measure the frequency and amplitude of the resulting motion of the spherical terminal 2080 of that body 2030, and a data acquisition computer to collect all resulting information. Another suitable testing method may include the use of suitable Viscometer machine, such as those used in rheology to test the performance of fluids under different frequencies and amplitudes of loading and under different temperatures.

The damping performance profile(s) may be optimized for the particular involuntary hand motions experienced by a particular user. The damping performance profile(s) may also be optimized for damping involuntary hand motions that occur within a given range of frequencies. In a particular example, the damping performance profile(s) may be optimized for involuntary hand motions occurring at a frequency falling within the range of 2 hz to 12 hz. A majority of the most common types of involuntary hand motions may occur in the range of 2 hz to 12 hz.

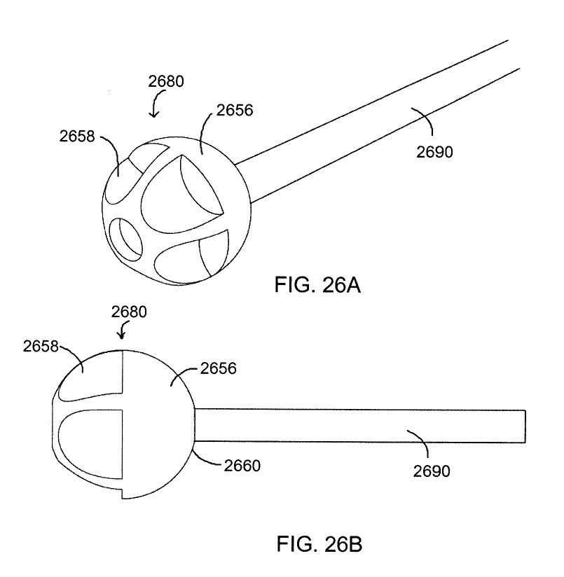

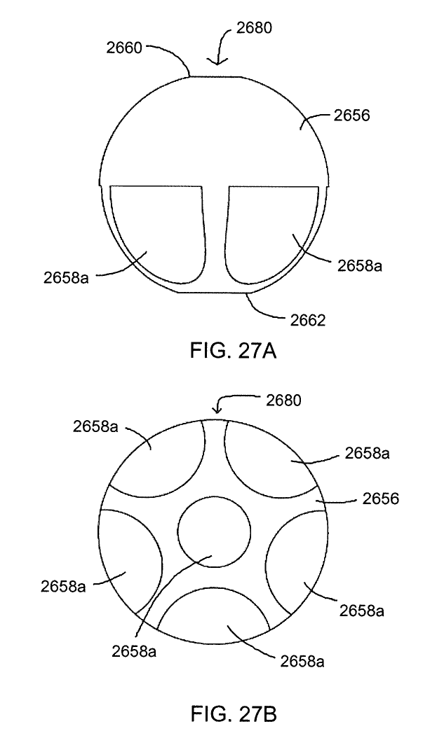

Reference is now made to FIGS. 26A to 27B which show an example of a spherical terminal 2680 with an outer surface 2656 including a plurality of cavities 2658. The spherical terminal 2680 may be used with the apparatus in either the first version of the third embodiment or the second version of the third embodiment, without modification to any previously described elements.

The spherical terminal 1380 (as shown in FIG. 13) and the spherical terminal 2080 (as shown in FIG. 20) are configured as perfectly spherical balls. When the spherical terminal 1380, for example, is a perfectly shaped spherical ball, a hardening reaction between the spherical terminal 1380 and the sheer thickening fluid (not shown) may only occur at small oscillation amplitudes. If tremors occur at high oscillation amplitudes, the sheer thickening fluid may begin to break since the sheer thickening fluid surrounding the spherical terminal 1380 may be an extremely thin layer (not shown). As a result, the spherical terminal 1380 may not be able to grip the sheer thickening fluid. The addition of a plurality of cavities 2658 to the outer surface 2656 can provide gripping points to increase sheer friction between the spherical terminal 2680 and the sheer thickening fluid. In this way, the spherical terminal 2680 can be configured to provide optimized shear friction for oscillation frequencies in the range of 2 hz to 12 Hz over a range of amplitudes.

Referring now to FIGS. 26A and 26B, the spherical terminal 2680 may be rigidly fixed to a link 2690 at a front end 2660. Any suitable combination of known methods and components may be used to implement this fixation. The outer surface 2656 may include with any number of cavities 2658 suitable to increase sheer friction between the terminal 2680 and the sheer thickening fluid. For example, the outer surface 2656 may include between 1 and 50 cavities 2658 to increase sheer friction between the spherical terminal 2680 and the sheer thickening fluid.

Referring now to FIGS. 27A and 27B shows a preferred embodiment of the spherical terminal 2680 in which the outer surface 2856 includes five cavities 2658a and one cavity 2658b. The five cavities 2658a are equally spaced apart around the outer surface 2656 of the spherical terminal 2680. Cavity 2658b is positioned at a rear end 2662 of the spherical terminal 2680. In an alternative embodiment (not shown), the outer surface 2656 may include nine cavities 2658a around the outer surface 2656 of the spherical terminal 2680. In still further embodiments (not shown), the size and spacing of each cavity 2658 may vary, and the number of cavities 2658 may also vary.

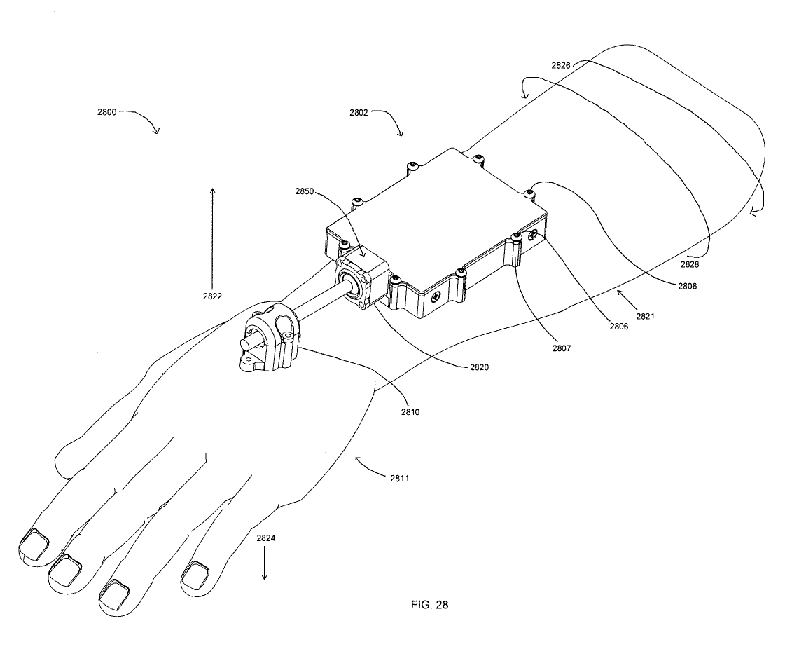

Reference is now made to FIGS. 28 to 31 which show a tuned mass damper 2802 for dampening involuntary forearm motions attached to an apparatus 2800 for damping involuntary hand motion.

As shown in FIG. 28, the apparatus 2800 may comprise a first portion 2810 securable to a hand 2811 and a second portion 2820 securable to a forearm 2821 and movable relative to the first portion 2810. The apparatus 2800 may comprise a body 2850 secured to the second portion 2820. The body 2850 may dampen the involuntary hand motions in one of at least a first direction 2822 and a second direction 2824.

The apparatus may further comprise the tuned mass damper 2802 secured to the second portion 2820. The tuned mass damper 2802 may dampen involuntary hand motions in one of at least a third direction 2826 and a fourth direction 2828. In this way, the apparatus 2800 is now designed to dampen the involuntary hand motions in at least the first direction 2822 and the second direction 2824 and involuntary forearm motions in at least the third direction 2826 and the fourth direction 2828, preferably without restricting voluntary hand or forearm motions in any direction. The third direction 2826 and the fourth direction 2828 may correspond to elbow supination and elbow pronation, respectively.

Referring still to FIG. 28, an example tuned mass damper 2802 is shown for dampening involuntary forearms movements in at least a third direction 2826 and a fourth direction 2828, the third direction 2826 being opposite the fourth direction 2828. The example tuned mass damper 2802 shown is attached to an apparatus 2800 for dampening involuntary hand motion. It should be apparent to one skilled in the art that the tuned mass damper 2802 can be attached to any one of the apparatuses for dampening involuntary hand motions described herein.

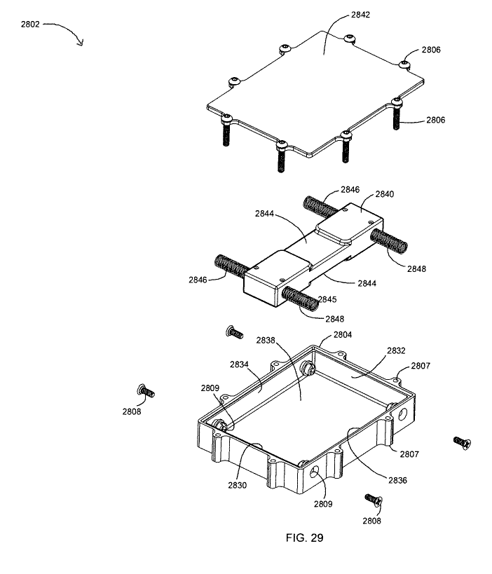

Referring now to FIG. 29, the tuned mass damper 2802 includes a housing 2804 securable to the second portion 2020 of the apparatus 2800 (shown in FIG. 28). The housing 2804 may be secured to the second portion 2020 either removably or permanently, using any suitable securement. Examples of suitable securements may include any one or a combination of sewing, gluing, and mechanical mechanisms for removable securement such as pairs of mating securement members. As shown in FIG. 28, an example tuned mass damper 2802 may be attached to the second portion 2020 by means of threaded inserts 2806 into preformed threaded openings 2807.

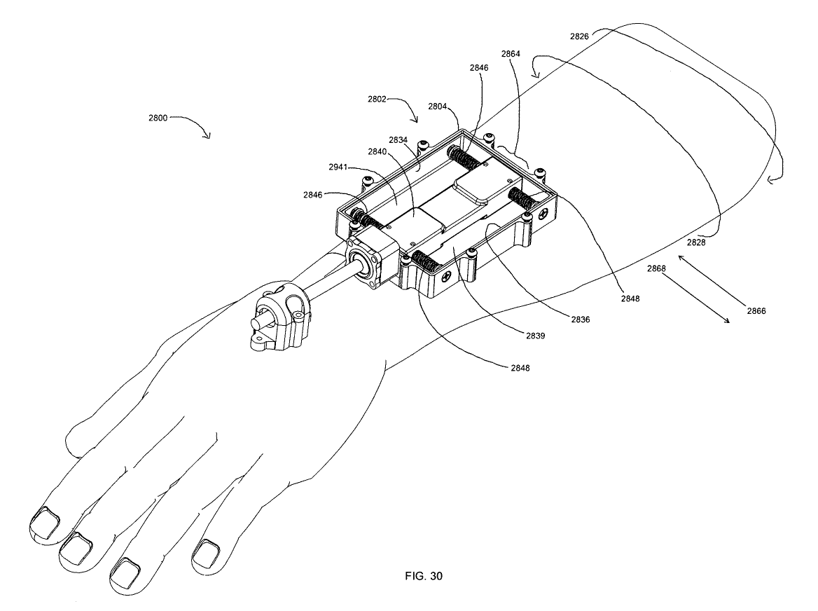

Referring again to FIG. 29, the housing 2804 may define an interior space 2838. The interior space 2838 may include a first interior wall 2830 positioned opposite to a second interior wall 2832 and a third interior wall 2834 positioned opposite to a fourth interior wall 2838. The tuned mass damper 2802 may further include an eccentric mass 2840 in the interior space 2838. The eccentric mass 2840 may extend between the first interior wall 2830 and the second interior wall 2832, dividing the interior space 2838 on adjacent sides of the eccentric mass 2840 into a first portion 2839 (shown in FIG. 30) and a second portion 2841 (shown in FIG. 30).

Referring still to FIG. 29, the tuned mass damper 2802 may further include a fluid (not shown) which may be added to a remaining volume (not shown) of the interior space 2838. The fluid may be any suitable fluid with a viscosity that may permit motion of the eccentric mass 2840 within the interior space 2838. In a preferred embodiment, the viscosity of the fluid which is used may remain stable with variations in temperature thereby allowing the tuned mass damper 2802 to operate consistently at different temperatures and use rates. For example, the fluid which is used in the tuned mass damper 2802 may include silicone oils, motor oils, polypropylene glycol, and polyethylene glycol.

The fluid may be added to the remaining volume such that the remaining volume is between 30 and 100% capacity. Drag forces between the motion of the eccentric mass 2840 and the fluid increase with the amount of the fluid. As a result, when the remaining volume is near 100% capacity, drag forces may significantly slow the motion of the eccentric mass 2840. In a preferred embodiment, the fluid may be added such that the remaining volume is between 50 and 80% capacity. When the fluid is added such that the remaining volume is between 50 and 80% capacity, drag forces may be within an optimal range to allow the eccentric mass 2840 to move out of phase with tremulous forearm motion.

The tuned mass damper 2802 further includes a top cover 2842 that may seal the fluid and the eccentric mass 2840 in the interior space 2838. The top cover 2842 may be secured to the housing 2804 either removably or permanently, using any suitable securement. Removably securing the top cover 2842 to the housing 2804 may provide easy access to the interior space 2838 if maintenance is required. As shown in FIG. 29, an example top cover 2842 may be attached to the housing 2804 by means of threaded inserts 2806 into preformed threaded openings 2807.

Referring still to FIG. 29, the eccentric mass 2840 may include at least one canal 2844 to permit the fluid to flow between the first portion 2839 and the second portion 2841 if the eccentric mass 2840 moves toward the third interior wall 2834 or the fourth interior wall 2836. Without the at least one canal 2844, the movement of the eccentric mass 2840 may be restricted since the flow of the fluid between the first portion and the second portion may not be permitted to occur. In this way, the eccentric mass 2840 can freely move out of phase with involuntary forearm oscillations in a linear direction 2866 and 2868 between the third interior wall 2834 and the fourth interior wall 2836, respectively.

The eccentric mass 2840 may include any number of canals 2844 suitable to permit the fluid to flow between the first portion 2839 and the second portion 2841, bearing in mind that each canal 2844 may reduce the mass of the eccentric mass 2840. For example, the eccentric mass 2840 may include between 1 and 50 canals. Referring to FIG. 29 shows an example eccentric mass 2840 in which the eccentric mass 2840 includes two canals 2844. In an alternative embodiment (not shown), the eccentric mass 2840 may include six canals 2844. In still further embodiments (not shown), the size and position of each canal 2844 may vary, and the number of canals 2844 may also vary.

Referring still to FIG. 29, the eccentric mass may include at least one spring 2846 that extends toward the third interior wall 2834 and at least one spring 2848 that extends toward the fourth interior wall 2836. Each spring 2846 and 2848 may be attached to the third interior wall 2834 and the fourth interior wall 2836, respectively, using any suitable securement. The springs 2846 and 2848 may bias the eccentric mass in a central region 2864 of the interior space 2838. The springs 2846 and 2848 may further improve the predictability of the tuned mass damper 2802 and may ensure the eccentric mass 2840 moves linearly within the interior space 2838.

Referring to FIG. 29, shows an example eccentric mass 2840 in which the eccentric mass 2840 includes two springs 2846 and two springs 2848. As shown, the two springs 2846 and the two springs 2848 may connect to the third interior wall 2834 and the fourth interior wall 2836, respectively, by means of threaded inserts 2808 into preformed threaded openings 2809. In an alternative embodiment (not shown), the eccentric mass 2840 may include six springs 2846 and six springs 2848. In still further embodiments (not shown), the size and location of each spring 2846 and 2848 may vary, and the number of springs 2846 and 2848 may also vary.

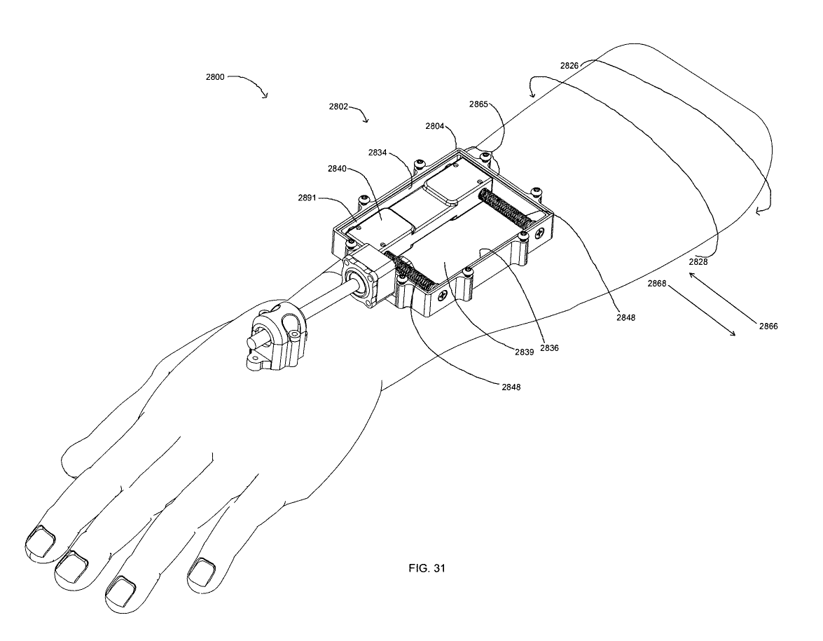

Referring to FIGS. 30 and 31 shows the tuned mass damper 2802 of FIG. 28 with the top cover 2842 (shown in FIG. 29) removed from the housing 2804.

Referring specifically to FIG. 30 shows the eccentric mass 2840 biased in the central region 2864 of the interior space 2838 by two springs 2846 extending to the third interior wall 2834 and two springs 2848 extending to the fourth interior wall 2836. When the eccentric mass 2840 is positioned in the central region 2854, the first portion 2839 and the second portion 2841 are approximately equal in size. An involuntary forearm movement in the third direction 2826 may cause the eccentric mass 2840 to move in a linear direction 2866 toward the third interior wall 2834. An involuntary forearm movement in the fourth direction 2828 may cause the eccentric mass 2840 to move in a linear direction 2868 toward the fourth interior wall 2836.

Referring specifically to FIG. 31 shows the eccentric mass 2840 positioned near the third interior wall 2834, in a stabilizing position 2865, to dampen forearm movement that has occurred in the third direction 2826. When the eccentric mass 2840 is in the stabilizing position 2865, the first portion 2839 is larger than the second portion 2941. The springs 2848 are shown in tension while the springs 2846 (shown in FIG. 30) are in compression. In this way, the springs 2846 and 2848 may allow the eccentric mass 2840 to move out of phase with the oscillation of involuntary forearm movements. In moving in the linear directions 2866 and 2868 between the third interior wall 2834 and the fourth interior wall 2836, the eccentric mass 2840 may act to stabilize tremulous forearm motion in both the third direction 2826 and the fourth direction 2828.

In an alternative embodiment (not shown), the tuned mass damper 2802 may be configured such that the motion of the eccentric mass 2840 within the interior space 2838 is non-linear. For example, the tuned mass damper 2802 may be arc-shaped as to conform to the contours of the forearm 2021. In this configuration, movement of the eccentric mass 2840 may directly follow elbow pronation and elbow supination movement.

The next paragraphs describe additional suitable materials, methods of manufacture, and embodiments or variations of the apparatus.

In some embodiments, the first and second portions may be portions of a glove. In these embodiments, the first and second portions may be manufactured to be secured removably or permanently to the glove. The terms removably or permanently may be taken to have similar meanings as described earlier for the same terms in relation to the body 1330. In some embodiments, including embodiments where the first and second portions are portions of a glove, the first and second portions may be manufactured integral to or separate from each other.

The apparatus may be manufactured to have a sufficiently small geometry relative to the hand and the forearm for at least a portion of the apparatus to be concealable under a garment worn by a user of the apparatus. In some cases, the body of the apparatus may be manufactured to be permanently or selectively concealed in the first and second portions. In embodiments where the apparatus includes a glove, the body may be manufactured to be permanently or selectively concealed in the glove. In other embodiments, a stand-alone glove may be provided separate from and not meant to be secured to the first and second portions. The stand-alone glove may be configured to conceal at least a part of each of the first and second portions and the body.

In some embodiments, the first and second portions may include collars or belt-type securing portions. In other embodiments, the first and second portions may be manufactured as stand-alone portions having an adhesive layer on at least one side of the portions for securing the portions to the hand and the forearm, respectively.

The apparatus may be manufactured using any combination of known materials and manufacturing and assembly methods suitable for each particular embodiment of the apparatus. For example, the glove may be made from any materials which will make the glove sufficiently elastic to allow for comfortable hand motions and at the same time will compress the hand and the forearm sufficiently to provide for acceptable levels of slack and of movements of the components of the apparatus. Levels of slack and of movements of the components may be unacceptable where, for example, the glove materials are chosen such that the forces resulting from involuntary hand motions will deform the glove instead of moving the terminal of the body. Desired levels of slack and of movements of the components may be dictated by particular demands for comfort of particular users of the apparatus. The glove may also be soft against the skin, have good chemical resistance, and be free from water absorption as these features may assist user comfort, durability, and functionality.

Materials for the glove may be, for example, selected to include any one or a combination of materials chosen from the following group: Spandex, Cotton, Coolmax, Thermoplastics, Polyspandex, Nylon, Bamboo, Neoprene, Vinyl, Terry foam, and contour foam.

The casing, the link, the terminal, and all other components of the first and second portions of the body may each be made from any known suitable material such as metal, rubber, plastic, or other materials, so long as the materials in combination provide for the functionality described in this document. In some cases, the components of the body may be individually or integrally 3D-printed, cast, or injection molded.

The body may be secured to the second portion either removably or permanently, using any suitable securement. Examples of suitable securements may include any one or a combination of sewing, gluing, and mechanical mechanisms for removable securement such as pairs of mating securement members. In some embodiments, insulation (not shown) may be attached around the body. In embodiments where the apparatus includes a glove, the insulation may be part of the glove. The insulation may slow down the rates at which the various components of the apparatus may experience temperature changes in response to varying ambient temperature conditions. Slower rates of temperature changes may provide for a more stable operation of the apparatus.

A number of embodiments have been described herein. However, it will be understood by persons skilled in the art that other variants and modifications may be made without departing from the scope of the embodiments as defined in the claims appended hereto. A person skilled in the art will also recognize that the embodiments described above should be read as representative of a plethora of permutations not explicitly described, said permutations incorporating elements from various embodiments.

1. An apparatus for damping involuntary hand motions comprising:
a first portion securable to a hand;
a second portion securable to a forearm and movable relative to the first portion;
at least one body comprising:
a casing defining an exterior surface and a spherical interior space having a port extending from the exterior surface into the spherical interior space, the casing directly connected to either the first portion or the second portion;
a connector comprising:
a spherical terminal filling the spherical interior space save for a gap between the spherical terminal and a wall of the spherical interior space; and,
a link passing through the port and connecting the spherical terminal to the portion of the first and second portions to which the casing is not directly secured; and,
a shear thickening fluid filling the gap between the spherical terminal and the wall of the spherical interior space, the shear thickening fluid having a viscosity that increases when the shear rate of the spherical terminal increases.

2. The apparatus of claim 1, wherein the link comprises a telescoping cylinder with one main stage and at least one additional stage.

3. The apparatus of claim 1, wherein the link comprises a shaft and a linear bearing.

4. The apparatus of claim 1, wherein the link comprises a shaft and a linear ball bearing.

5. The apparatus of claim 1, where the gap between the spherical terminal and the wall of the spherical interior space is between 0.0746 mm and 1.0254 mm.

6. The apparatus of claim 1, wherein the shear thickening fluid has a composition selected to resist involuntary hand motions above 3 hertz.

7. The apparatus of claim 1, wherein the shear thickening fluid has a composition selected to resist involuntary hand motions above 2 hertz.

8. The apparatus of claim 1, wherein the shear thickening fluid has a composition selected not to resist hand motions below 1 hertz.

9. The apparatus of claim 1, wherein the shear thickening fluid has a composition selected not to resist hand motions below 2 hertz.

10. The apparatus of claim 1, further comprising an elastic o-ring positioned between the casing and the spherical terminal to hinder the passage of shear thickening fluid through the port.

11. The apparatus of claim 1, wherein the spherical terminal is configured to include an outer surface comprising a plurality of cavities.

12. The apparatus of claim 1, further comprising a tuned mass damper for damping involuntary forearm motions, the tuned mass damper comprising:
a housing securable to the second portion, the housing defining an interior space which comprises a first interior wall positioned opposite to a second interior wall and a third interior wall positioned opposite to a fourth interior wall;
an eccentric mass in the interior space, the eccentric mass extending between the first interior wall and the second interior wall, dividing the interior space adjacent to the eccentric mass into a first portion and a second portion; and
a fluid which is added to a remaining volume of the interior space;
wherein
the eccentric mass comprises at least one canal to permit the fluid to flow between the first portion and the second portion; and
the eccentric mass is free to move out of phase with involuntary forearm oscillations in a linear direction between the third interior wall and the fourth interior wall.

13. The apparatus of claim 12, wherein the eccentric mass is biased in a central region of the interior space by a plurality of springs that connect the third interior wall with the eccentric mass and by a plurality of springs that connect the fourth interior wall with the eccentric mass such that the eccentric mass can move out of phase with involuntary forearm oscillations in a linear direction between the third interior wall and the fourth interior wall.

14. An apparatus for damping involuntary hand motions occurring in at least first and second directions, the first direction being opposite the second direction, the apparatus comprising:
a first portion securable to a hand;
a second portion securable to a forearm and movable relative to the first portion; and
at least one body secured to the second portion and comprising
an exterior surface, an interior space containing a Non-Newtonian fluid, a port extending from the exterior surface into the interior space,
a terminal in the interior space movable between the first and the second positions and biased toward the first position, the first position being located farther away from the first portion than the second position, and
a link passing through the port and connecting the terminal to the first portion;
wherein
when the first portion is secured to the hand and the second portion is secured to the forearm, the link is in tension, and
the involuntary hand motions in the first direction move the terminal from the first position toward the second position.

15. The apparatus of claim 14, wherein the link is configured to oppose lengthwise tensile forces and to substantially not oppose lengthwise compression forces.

16. The apparatus of claim 14, wherein the Non-Newtonian fluid has a composition selected to achieve a damping performance profile of the least one body optimized for damping involuntary hand motions ranging in frequency from 2 to 12 hertz.

17. The apparatus of claim 14, wherein the terminal divides the interior space into two sections and comprises:
a resistance flow opening connecting the first and second sections for bidirectional flow of the Non-Newtonian fluid; and
a check valve connecting the first and second sections for unidirectional flow of the Non-Newtonian fluid.

18. The apparatus of claim 14, further comprising a tuned mass damper for damping involuntary forearm motions, the tuned mass damper comprising:
a housing securable to the second portion, the housing defining an interior space which comprises a first interior wall positioned opposite to a second interior wall and a third interior wall positioned opposite to a fourth interior wall;
an eccentric mass in the interior space, the eccentric mass extending between the first interior wall and the second interior wall, dividing the interior space adjacent to the eccentric mass into a first portion and a second portion; and
a fluid which is added to a remaining volume of the interior space;
wherein
the eccentric mass comprises at least one canal to permit the fluid to flow between the first portion and the second portion; and
the eccentric mass is free to move out of phase with involuntary forearm oscillations in a linear direction between the third interior wall and the fourth interior wall.

19. The apparatus of claim 18, wherein the eccentric mass is biased in a central region of the interior space by a plurality of springs that connect the third interior wall with the eccentric mass and by a plurality of springs that connect the fourth interior wall with the eccentric mass such that the eccentric mass moves out of phase with involuntary forearm oscillations in a linear direction between the third interior wall and the fourth interior wall.

20. The apparatus of claim 14, wherein:
the least one body of claim 14 is a first body,
the apparatus includes a second body as claimed in claim 14,
the second body is secured to the second portion, and
when the first portion is secured to the hand and the second portion is secured to the forearm, the link of the second body is in tension, and
the involuntary hand motions in the second direction move the terminal of the second body from the first position of the terminal of the second body toward the second position of the terminal of the second body.
